(19) United States
(12) Patent Application Publication
(10) Pub. No.: US 2023/0031287 A1
(43) Pub. Date: Feb. 2, 2023

(54) METHOD AND COMPOSITION FOR ENHANCING THE IMMUNE RESPONSE

(71) Applicant: Northwestern University, Evanston, IL (US)

(72) Inventors: Pablo Penaloza-MacMaster, Oak Park, IL (US); Nicole Palacio, Evanston, IL (US)

(21) Appl. No.: 17/784,891

(22) PCT Filed: Dec. 14, 2020
(86) PCT No.: PCT/US2020/064941

§ 371 Date: Jun. 13, 2022

Related U.S. Application Data
(60) Provisional application No. 62/947,953, filed on Dec. 13, 2019.

(51) Int. Cl. A61K 39/395; A61K 39/145; A61K 39/29; A61K 39/12; A61K 39/25; A61K 39/275; A61K 39/165; A61K 39/205; A61K 39/13; A61K 39/20; A61K 39/21; A61P 31/16; A61P 31/18; A61P 31/14; A61P 31/20

(52) U.S. Cl. CPC A61K 39/3955; A61K 39/145; A61K 39/29; A61K 39/292; A61K 39/12; A61K 39/25; A61K 39/275; A61K 39/165; A61K 39/205; A61K 39/13; A61K 39/20; A61K 39/21; A61P 31/16; A61P 31/18; A61P 31/14; A61P 31/20; A61K 2039/5256

(57) ABSTRACT

Disclosed herein are methods, compositions, and kits useful to enhance an immune response against an antigen and to improve vaccine efficacy. The disclosed methods, compositions, and kits may be utilized to improve vaccine immunogenicity and enhance immune protection following subsequent antigen challenges. In some embodiments, the methods include co-administering a blocker of the IFN-I pathway with an antigen that is used as part of a vaccine, such as a viral vaccine.

### CROSS-REFERENCE TO RELATED APPLICATIONS

This application claims the benefit of U.S. Application No. 62/947,953 filed on Dec. 13, 2019, which is incorporated herein by reference in its entirety.

### STATEMENT REGARDING FEDERALLY SPONSORED RESEARCH OR DEVELOPMENT

This invention was made with government support under AI132848 and AI117943 awarded by National Institutes of Health. The government has certain rights in the invention.

### FIELD

The field of the invention relates to methods and compositions for enhancing immune responses against antigens and vaccination. The disclosed methods typically include a step of transiently blocking the activity of interferon in order to enhance immune responses against antigens and vaccination.

### BACKGROUND

Type I interferons (IFN-I) are a first line of defense against viral infections. These molecules are induced within hours of infection, and prior studies demonstrate that IFN-I can exert immunostimulatory, as well as immunosuppressive effects. For example, IFN-I help to prime adaptive immune responses following acute viral infections and vaccinations, but constitutive IFN-I production during chronic viral infections can impair adaptive immune responses. Prior studies have interrogated the effect of protracted absence of IFN-I signaling after viral immunization, using genetic knockout models or long-term IFN-I blockade. These earlier studies have shown that long-term IFN blockade impairs immune responses and virus clearance. However, the effect of short-term IFN-I blockade especially at the time of immunization has remained understudied. Our study demonstrates that short-term IFN-I blockade results in a significant improvement of immune responses following vaccination.

### SUMMARY

Disclosed herein are methods, compositions, and kits useful to enhance or improve an immune response against an antigen and to improve vaccine efficacy. The disclosed methods, compositions, and kits may be utilized to improve vaccine immunogenicity and enhance immune protection following subsequent antigen challenges. In some embodiments, the methods include co-administering an IFN blocking agent with an antigen that is used as part of a vaccine, such as a vaccine against an infectious agent, such as viral vaccine or bacterial vaccine. In some embodiments, the vaccine is against a disease such as cancer. In some embodiments, the vaccine comprises one or more of: an influenza vaccine, a hepatitis A vaccine, a hepatitis B vaccine, a human papilloma virus vaccine, a zoster vaccine, a smallpox vaccine, a measles vaccine, a rabies vaccine, a poliovirus vaccine, a Japanese encephalitis vaccine, a rubella vaccine, a rotavirus vaccine, a yellow fever vaccine, a varicella virus vaccine, an HIV vaccine, and other replicating viral vaccines, and combinations thereof.

In some embodiments, the IFN blocking agent comprises and IFN-I receptor blocking antibody. For example, in some embodiments, and IFN-I receptor blocking antibody comprises αIFNAR1 clone MAR1-5A3, and/or anifrolumab.

In some embodiments, the vaccine and the IFN blocking agent are administered simultaneously; in some embodiments, the IFN blocking agent is administered separately from the antigen, and is administered, for example, within 24 hours, within 48 hours, or within 72 hours of administering the pharmaceutical composition comprising the effective amount of the antigen.

In some embodiments, an improved immune response is detected in a subject treated according to the methods disclosed herein; such and improved immune response may include one or more of improved vaccine immunogenicity and/or enhanced immune protection; an increase in antigen-specific CD8 T-cells; an increase in antigen-specific CD4 T-cells; an increase in antigen-specific B cells or antibodies; an increase in heterologous antibodies; a lack of, or reduction in, disease symptoms; and/or a lack of, or reduction in the antigen. Such an improved immune response may be detected in the subject after the subject further subsequently is administered the antigen and/or the subject is later challenged with an infectious agent (e.g. a virus or a bacteria) or disease (e.g., cancer) comprising the antigen.

Also disclosed herein are immunogenic composition comprising an effective amount of an antigen and a therapeutic agent that blocks the activity of IFN-1. In some embodiments, the immunogenic composition comprises a vaccine against an infectious agent that comprises the antigen. In some embodiments, the vaccine comprises one or more of: an influenza vaccine, a hepatitis A vaccine, a hepatitis B vaccine, a human papilloma virus vaccine, a zoster vaccine, a smallpox vaccine, a measles vaccine, a rabies vaccine, a poliovirus vaccine, a Japanese encephalitis vaccine, a rubella vaccine, a rotavirus vaccine, a yellow fever vaccine, a varicella virus vaccine, an HIV vaccine, and other replicating viral vaccines, and combinations thereof. In some embodiments, the vaccine comprises an anticancer vaccine. In some embodiments, the IFN blocking agent comprises and IFN-I receptor blocking antibody. For example, in some embodiments, and IFN-I receptor blocking antibody comprises αIFNAR1 clone MAR1-5A3, and/or anifrolumab.

Also disclosed herein are kits comprising (a) an antigen, optionally wherein the kit comprises a vaccine; and (b) an IFN-I blocking agent. In some embodiments, the kit comprises a viral vaccine, or an anti-cancer vaccine, and comprise and IFN-I receptor binding antibody.

### BRIEF DESCRIPTION OF THE FIGURES

FIG. 1A-J. IFN-I blockade improves immunological memory after acute viral infection. (A) Experimental approach for inducing a short-term blockade of IFN-I after acute viral infection in C57BL/6 mice. (B) Duration of IFNAR1 blockade. PBMCs were stained with a fluorescently labeled αIFNAR1 antibody to evaluate competitive binding. Dotted line represents the limit of detection (IFNAR1 staining in Ifnar1−/− mice). MFI, mean fluorescence intensity. (C) Summary of Zika-specific CD8 T cells. (D) Summary of Zika-specific antibody responses. (E) Representative FACS plots showing the frequencies of IFNγ+CD8 T cells after stimulation with Zika peptide. (F) Representative FACS plots showing the frequencies of polyfunctional CD8 T cells after stimulation with Zika peptide. (G) Summary of double cytokine producer CD8 T cells after stimulation with Zika peptide. (H) Summary of triple cytokine producer CD8 T cells after stimulation with Zika peptide. Data in panels E-H are from week 2 after infection with Zika PRV strain. Zika-specific CD8 T cells were enumerated after 5-h stimulation with a Zika-specific peptide (IGVSNRDFV). (I) Summary of GP33-specific CD8 T cells after coronavirus infection (MHV-GP33). (J) Summary of coronavirus (MHV)-specific antibody responses. Bottom lines in panels D and J indicate limit of detection. Data represent two or more combined experiments; n=4-5 per each independent experiment. Zika infections were with 104 PFU/mouse, and coronavirus infections were with 102 PFU/mouse. **, P<0.01; ***, P<0.001 by the Mann-Whitney U test. Error bars represent SEM.

FIG. 2A-L. IFN-I blockade improves immunological memory after viral vaccination. C57BL/6 mice were immunized intramuscularly with 104 PFU of the indicated vaccines mixed with control antibodies or IFNAR1-blocking antibodies, similar to FIG. 1A. (A) Representative FACS plots showing frequencies of YFV specific (IFNγ+) CD8 T cells after 5-h peptide stimulation (IGITDRDFI). (B) Summary of YFV-specific CD8 T cells. (C) Summary of YFV-specific antibody responses. (D) Representative FACS plots showing the frequencies of OVA-specific (KbSIINFEKL+) CD8 T cells. (E) Summary of OVA-specific CD8 T cells. (F) Summary of OVA-specific antibody responses. (G) Representative FACS plots showing the frequencies of SIV-specific (DbAL11+) CD8 T cells. (H) Summary of SIV-specific (DbAL11+) CD8 T cells. (I) Summary of SIV-specific antibody responses. (J) Representative FACS plots showing the frequencies of HIV-specific (IFNγ+) CD8 T cells after 5-h peptide stimulation (BG505 envelope peptide pools). (K) Summary of HIV-specific CD8 T cells. (L) Summary of HIV-specific antibody responses (BG505, Glade A envelope). FACS plots are gated from total CD8 T cells at week 2 after infection. Dotted lines indicate limit of detection. Experiments were performed two to four times; n=5 mice per experiment (data from all experiments are shown). *, P<0.05; **, P<0.01; ***, P<0.001 by the Mann-Whitney U test. Error bars represent SEM.

FIG. 3A-P. Transcriptional analyses of virus-specific CD8 T cells. C57BL/6 mice were immunized intramuscularly with 10⁴ PFU of acute LCMV mixed with control antibodies or αIFNAR1 antibodies similar to FIG. 1A, and gene expression was assessed on virus-specific CD8 T cells after 7 d. (A) Experimental approach for performing RNA-Seq. Splenic CD8 T cells were MACS purified by negative selection, followed by FACS sorting of live, CD8+, CD44+, andDbGP33+ cells. This resulted in ˜97% pure population of virus-specific CD8 T cells for transcriptional analyses. (B) DbGP33+CD8 T cell purity test. (C) Volcano plot showing differentially expressed genes between mice that received IgG or αIFNAR1 at the time of infection. (D) Heat map showing the top genes up-regulated in αIFNAR1. (E) Validation of CD44 expression at the protein level by flow cytometry. MFI, mean fluorescence intensity. (F) IPA. (G) GSEA of T cell costimulation—driven genes. The GSE26669 signature contains genes that are normally increased after T cell costimulation. (H) GSEA of IFNα stimulation signature. (I) GSEA of dual IFNα/β stimulation signature. (J) Validation of IFN-I levels at the protein level using ELISA. (K) GSEA of IFNγ stimulation signature. (L) GSEA of IL-12 stimulation signature. (M) GSEA of IL-15 stimulation signature. (N) GSEA of CD8 T cell activation signature. (0) GSEA of effector CD8 T cell signature. RNA-Seq experiments were performed once, using four mice in the control group and five mice in the αIFNAR1 group. *, P<0.05; **, P<0.01; ***, P<0.001 by the Mann-Whitney U test. Error bars represent SEM. FDR, false discovery rate; NES, normalized enrichment score; ES, enrichment score. (P) is a radar plot analysis showing enrichment in INF-induced genes in CD8 T cells after transient IFN-I blockade. Panels inside the box represent RNA-Seq data at day 7. Other panels represent confirmatory data at the protein level.

FIG. 4A-K. Phenotypic and functional analyses of DC responses. (A) Murine DCs were infected with LCMV-GFP in the presence of control antibodies or αIFNAR1 antibodies. (B) DCs infected with coronavirus (MHV-GFP) in the presence of control antibodies or αIFNAR1 antibodies. DCs were imaged by immunofluorescence 72 h after infection (see Materials and methods). Green (GFP) indicates viral foci and DAPI (blue) indicates cell nuclei. (A and B) Scale bars are 100 (C) Experimental approach for analyzing DC responses in vivo. C57BL/6 mice were immunized intramuscularly with 10⁴ PFU of LCMV-OVA mixed with control antibodies or αIFNAR1 antibodies, similar to FIG. 1A. After 5 d, splenic DCs were characterized. (D) Representative FACS plots showing DCs that present cognate antigen in the context of MHC-I (KbSIINFEKL+). FACS plots gated on DCs (live, NK1.1−, CD3−, CD19−, Ly6G−, and CD11c+). (E) Summary of DCs that present cognate antigen. (F) Costimulatory CD80 expression on DCs. (G) Costimulatory CD86 expression on DCs. (H) Experimental approach to interrogate the DC-intrinsic effects of IFN-I. Wild-type or Ifnar1−/− DCs were infected with LCMV, and after 1 d, 2.5×10⁵ DCs were intravenously transferred into naive mice to measure CD8 T cell priming. (I) Summary of LCMV DbGP33—(left panel) and DbNP396 (right panel)-specific CD8 T cell responses at day 7 after DC transfer. Experiments were performed two times; n=4-5 mice per experiment (data from one representative experiment are shown). Dotted lines represent limit of detection based on naive mean fluorescence intensity (MFI) levels. **, P<0.01; ***, P<0.001 by the Mann-Whitney U test. (J) show the experimental set up to interrogate DC function after hyper acute IFNAR1 blockade. For these in vitro DC studies, draining lymph nodes from 5 different mice were pooled, and DCs were MACS-purified and co-cultured with cell trace violet (CTV)-labeled Thy1.1+P14 cells to measure proliferation. Wells were run in duplicates. (K) Histograms showing CTV dilution at day 3 (gated on Thy1.1+D^{b}GP33+CD8 T cells). *, P≤0.05.

FIG. 5A-G. Memory T cell subset analyses after short-term IFN-I blockade. C57BL/6 mice were immunized intramuscularly with 10⁴ PFU of VSV-OVA mixed with control antibodies or αIFNAR1 antibodies, similar to FIG. 1A. Phenotypic characterization of virus-specific CD8 T cells was performed at day 60. (A) Number of VSV (KbRGY)-specific CD8 T cells in liver. (B) Number of VSV-specific CD8 T cells in spleen. (C) Representative FACS plots showing memory subsets on VSV-specific CD8 T cells from spleen. (D) Frequency of effector memory CD8 T cells (CD62L−/CD127+) in spleen. (E) Frequency of central memory CD8 T cells (CD62L+/CD127+) in spleen. (F) Number of effector memory CD8 T cells (CD62L−/CD127+) in spleen. (G) Number of central memory CD8 T cells (CD62L+/CD127+) in spleen. Experiment was performed two times (data from one representative experiment are shown). *, P<0.05; **, P<0.01; ***, P<0.001 by the Mann-Whitney U test. Error bars represent SEM.

FIG. 6A-K. IFN-I blockade during an initial viral prime improves future host protection following future reinfections. C57BL/6 mice were immunized intramuscularly with the indicated viruses mixed with control antibodies or αIFNAR1 antibodies, similar to FIG. 1A, and immune protection was assessed weeks later using various pathogen challenges. (A and B) Immune protection in Zika PRV-primed mice. (A) Viral titers in brain at day 3 following intracranial Zika MR766 challenge (10⁴ PFU). (B) Antibody-mediated Zika MR766 neutralization in sera 14 d following Zika PRV prime by plaque reduction neutralization titer (PRNT) assays. (C) Immune protection in coronavirus (MHV)-primed mice. Viral titers in lungs at day 3 following intranasal coronavirus challenge (10⁶ PFU). (D) Immune protection in YFV-17D-primed mice. Viral titers in sera at day 3 following intravenous YFV-17D challenge (10⁶ PFU). In this experiment, we first vaccinated wild-type mice with YFV-17D, and after 2 wk we transferred sera from these immune mice into naive Ifnar1−/− mice (which are highly susceptible to YFV-17D). 1 d later, the recipient Ifnar1−/− mice were challenged intravenously with YFV-17D. (E and F) Immune protection in VSV-OVA-primed mice. (E) Survival following intravenous supra-lethal challenge with 10⁷ CFU of LM-OVA. (F) Viral titers in lung at day 5 following intranasal challenge with 2×10⁶ PFU of VV-OVA. (G and H) Immune protection in LCMV-immunized mice. (G) Survival following intravenous supra-lethal challenge with 107 CFU of LM-GP33. (H) Bacterial titers in liver (˜36 h after LM-GP33 challenge). (I) Viral titers in lung at day 3 following intranasal challenge with 10⁶ PFU of coronavirus-GP33 (MHV-GP33). Limit of detection is 47 PFU. Dotted lines indicate limit of detection. Experiments were performed two times (data from all experiments are shown, except for panel C, which has data from only one experiment). The Mann-Whitney U test was used in most panels, except for panels E and G, which used the Mantel-Cox test. *, P<0.05; **, P<0.01; ***, P<0.001 by indicated statistical tests. Error bars represent SEM. (J) and (K) shows immune protection in mice vaccinated with rhabdovirus vector (VSV-OVA). (J) Left: Tumor free survival after B16-OVA melanoma challenge. (K) Right: Representative images of tumors after B16-OVA melanoma challenge (˜day 30). 10⁵ B16-OVA melanoma cells were injected subcutaneously.

FIG. 7A-M. Shows that MAR1-5A3 blocks IFNAR1 in vitro and in vivo as well as CD8, CD4, and antibody responses with an experimental HIV vaccine (LCMV-HIV). Effects of short-term IFN-I blockade on experimental SIV/HIV vaccines. (A) Validation of the IFNAR1 antibody (MAR1-5A3) in vitro. CT2A cells were incubated with 20 μg of IgG1 (MOPC-21) or IFNAR1-blocking antibody (MAR1-5A3) for 30 min before treatment with IFN-I (1,000 units) overnight. Representative histograms of MHC-I expression are shown. Note that MHC-I expression is induced by IFN-I signaling. (B) C57BL/6 mice were immunized intramuscularly with 104 PFU of LCMV-SIV mixed with 100 μg of control antibodies or IFNAR1-blocking antibodies, similar to FIG. 1A. Representative histograms of phosphorylated STAT1 (pSTAT1) in whole PBMCs 1 d after infection (naive levels represented by dashed histogram, which overlapped with the αIFNAR1 group). Note that pSTAT1 is induced by IFN-I signaling. (C) Representative FACS plots showing the frequencies of LCMV- and SIV-specific CD8 T cells (gated from live CD8+ lymphocytes) in spleen, lymph nodes, and liver. (D) Summary of LCMV- and SIV-specific CD8 T cells in spleen, lymph nodes, and liver. (E) In vitro SIV neutralization in LCMV-SIV-immunized mice; sera from IgG1- or αIFNAR1-treated mice were tested for their ability to neutralize SIV in vitro. Neutralization was measured by TZM-b1 assay. (F) Representative FACS plots showing the frequencies of germinal center (GC) B cells in draining lymph nodes (gated from live CD3− B220+ IgM− IgD− lymphocytes) at day 14. PNA, peanut agglutinin. (G) Levels of antibody that bind to heterologous HIV-1 (SF162, Glade B envelope) at day 14. (H) Weight of mice following LCMV-HIV vaccination. (I) Representative FACS plots showing the frequencies of HIV-specific CD8 (left) and CD4 (right) T cells (gated from live CD8+ or CD4+ lymphocytes). (J) Summary of HIV-specific CD8 T cells that are double (IFNγ+ TNFα+) producers. (K) Summary of HIV-specific CD8 T cells that are triple (IFNγ+ TNFα+IL-2) producers. (L) Summary of HIV-specific CD4 T cells that are double (IFNγ+ TNFα+) producers. (M) Summary of HIV-specific CD4 T cells that are triple (IFNγ+ TNFα+IL-2) producers. Data in panels C and D are from day 48 after infection. All other panels are from day 14 after infection. All experiments were performed at least twice with n=5-7 mice per group per experiment; data are from one representative experiment. *, P<0.05; **, P<0.01; ***, P<0.001 by the Mann-Whitney U test. Error bars represent SEM.

FIG. 8A-C. Show OVA-specific responses after VSV-OVA immunization using subcutaneous or intranasal routes. IFN-I blockade improves immunological memory following subcutaneous or intranasal infection. C57BL/6 mice were immunized subcutaneously (A and B) or intranasally (C) with 10⁴ PFU of VSV-OVA mixed with control antibodies or αIFNAR1. (A) Summary of OVA-specific CD8 T cells in PBMCs. (B) Summary of OVA-specific antibody responses in sera. (C) Summary of OVA-specific CD8 T cells in PBMCs at day 16 after infection. Data from panels A and B are combined from two experiments, with a total of eight to nine mice per group; data from panel C is from one experiment with four to five mice per group. Dotted line represents limit of detection. *, P<0.05; **, P<0.01; ***, P<0.001 by the Mann-Whitney U test. Error bars represent SEM.

FIG. 9A-I. Show that short-term IFNAR1 blockade increases hyperacute viral loads. Short-term IFN-I blockade transiently increases viral loads without abrogating viral clearance. C57BL/6 mice were immunized intramuscularly with escalating doses of the indicated viral vectors, mixed with control antibodies or αIFNAR1 antibodies, and viral loads were quantified at the site of infection (muscle) at different time points. (A) Viral loads at day 7 after LCMV-OVA infection. (B) Viral loads at day 7 after VSV-OVA infection. (C) Viral loads at day 7 after YFV-17D infection. (D) Viral loads at day 3 after LCMV-OVA infection. (E) Viral loads at day 3 after VSV-OVA infection. (F) Viral loads at day 3 after YFV-17D infection. Quadriceps were harvested at the indicated time points, and viral load was quantified by plaque assay. (G) Systemic viral load after intramuscular LCMV-OVA infection (104 PFU/mouse). This panel shows that very low viremia is detected after intramuscular infection. Undiluted sera were used to improve plaque assay sensitivity. (H) OVA-specific (KbSIINFEKL) CD8 T cells at day 15 after LCMV-OVA infection. (I) OVA-specific CD8 T cells at day 15 after VSV-OVA infection. Note that increasing vaccine dose does not significantly alter early viral loads and does not induce a commensurate increase in immunogenicity. Dotted lines represent limit of detection. Data are from two to three experiments, with four to five mice per group (all data are shown). *, P<0.05; **, P<0.01; ***, P<0.001 by the Mann-Whitney U test. Error bars represent SEM.

FIG. 10A-K. The adjuvant effect of short-term IFN-I blockade is time and virus replication dependent. (A-D) C57BL/6 mice were immunized intramuscularly with 10⁴ PFU of LCMV-SIV, and at day 5 after infection, mice were treated with control antibodies or αIFNAR1 antibodies intramuscularly. (A) Experimental approach for evaluating the effect of IFN-I blockade at day 5. (B) Summary of vector-specific (DbGP33+) CD8 T cell responses. (C) Summary of SIV-specific (DbAL11+) CD8 T cell responses. (D) Summary of antibody responses in sera at day 14. (E) VSV-specific IgG in sera after infection with 104 PFU of VSV (ΔG). (F) VSV-specific CD8 T cells (KbRGY+) after infection with 104 PFU of VSV (ΔG*G). (G) SIV-specific IgG after infection with 104 PFU of LCMV-SIV(ΔGP). (H) HIV-specific IgG in sera after infection with 109 viral particle of Ad5-HIV (ΔE1/E3). Data from panels E-H are from day 14 after infection. (I) Experimental approach for evaluating immune responses after acute viral infection with IFN-I supplementation. (J) LCMV-specific (DbGP33+) CD8 T cells in blood at day 7 after infection with LCMV-HIV. (K) VSV-specific (KbRGY+) CD8 T cells in blood at day 7 after infection with VSV-OVA. All data are from PBMCs or sera. All experiments were performed at least twice with four to five mice per group per experiment; results of a representative experiment are shown. For panels J and K, IFNα2 was administered intraperitoneally on days 0, 1, and 2 (5 μg/dose). All experiments were performed at least twice with four to five mice per group per experiment. **, P<0.01; ***, P<0.001 by the Mann-Whitney U test. Error bars represent SEM.

FIG. 11A-B. Show immune protection in a different genetic background (BALB/c). Short-term IFN-I blockade during viral prime improves anamnestic protection in BALB/c mice. BALB/c mice were immunized intramuscularly with 10⁴ PFU of LCMV-OVA mixed with control IgG antibodies or αIFNAR1. After 30 d, mice were challenged intranasally with 10⁶ PFU of VV-OVA, and viral loads were quantified in lungs at day 5 after challenge. (A) Experimental approach for evaluating whether IFN-I blockade improves vaccine-induced protection in a different host genetic background. (B) Viral loads in lungs at day 5 after challenge. Data are from one representative experiment with four mice. Experiment was performed twice with similar results. Dotted line represents limit of detection. *, P<0.05 by the Mann-Whitney U test.

### DETAILED DESCRIPTION

The present invention is described herein using several definitions, as set forth below and throughout the application.

Unless otherwise specified or indicated by context, the terms “a”, “an”, and “the” mean “one or more.” For example, “an antigen” should be interpreted to mean “one or more antigens.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the term “subject” may be used interchangeably with the terms “patient” or “individual” and means an animal, which may be a human or non-human animal. A “subject in need thereof” may include a subject having or at risk of disease or disorder. In some embodiments, such a disease or disorder may be prevented, or its symptoms, duration, or severity alleviated or attenuated, via vaccination. Individuals who are treated with the compositions disclosed herein may be at risk for infection with a virus or may have already been infected.

As used herein, the term “antigen” refers to an agent which is administered to a subject in need thereof in order to elicit an immune response against the antigen, which may include a protective immune response against the antigen such as in vaccination. Suitable antigens may comprise viruses, proteins (or polypeptides or peptides), carbohydrates, lipids, nucleic acid, and any combination thereof.

Viruses, Proteins, Polypeptides, and Peptides

As contemplated herein, viruses, proteins, polypeptides, and peptides may be administered as antigens. Proteins, polypeptides, and peptides may be described by an amino acid sequence (e.g., via SEQ ID NO). A variant of the disclosed proteins, polypeptides, and peptides may comprise an amino acid sequence having less than 100% sequence identify to a reference amino acid sequence and may have at least about 50%, 60%, 70%, 80%, 90%, 95%, 96%, 97%, 98%, or 99% sequence identity to a reference amino acid sequence. Variants may include polypeptides having one or more amino acid substitutions, deletions, additions and/or amino acid insertions relative to a reference polypeptide (e.g., relative to any of SEQ ID NOs:1-6), where optionally the variant of the reference polypeptide exhibits one or more biological activities associated with the reference polypeptide.

As used herein, the terms “amino acid” and “amino acid sequence” refer to an oligopeptide, peptide, polypeptide, or protein sequence (which terms may be used interchangeably), or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

The amino acid sequences contemplated herein may include conservative amino acid substitutions and/or non-conservative amino acid substitutions relative to a reference amino acid sequence. For example, a variant polypeptide may include conservative amino acid substitutions and/or non-conservative amino acid substitutions relative to the wild-type polypeptide. “Conservative amino acid substitutions” are those substitutions that are predicted to interfere least with the properties of the reference polypeptide. In other words, conservative amino acid substitutions substantially conserve the structure and the function of the reference protein. The following table provides a list of exemplary conservative amino acid substitutions.

| Original | |
|---|---|
| Residue | Conservative Substitutson |
| Ala | Gly, Ser |
| Arg | His, Lys |
| Asu | Asp, Gia, His |
| Asp | Asn, Glu |
| Cys | Ala, Ser |
| Gln | Asn, Glu, His |
| Glu | Asp, Arg, His |
| Gly | Ala |
| His | Asn, Arg, Gln, Glu |
| Ile | Leu, Val |
| Leu | Ile, Val |
| Lys | Arg, Gln, Glu |
| Met | Leu, ile |
| Ple | His, Met, Leu, Trp Tyr |
| Ser | Cys, Thr |
| Th | Ser, Val |
| Trp | Phe, Tyr |
| Tyr | His, Phe, Trp |
| Val | Ile, Len, Thr |

In contrast, “Non-conservative amino acid substitutions” are those substitutions that are predicted to interfere most with the properties of the reference polypeptide. For example, non-conservative amino acid substitutions may not conserve the structure and/or the function of the reference protein (e.g., substitution of a polar amino acid for a non-polar amino acid and/or substitution of a negatively charged amino acid for a positively charged amino acid).

A “deletion” refers to a change in the amino acid or nucleotide sequence that results in the absence of one or more amino acid residues or nucleotides relative to a reference sequence. A deletion may remove at least 1, 2, 3, 4, 5, 10, 20, 50, 100, or 200 amino acids residues or nucleotides. A deletion may include an internal deletion or a terminal deletion (e.g., an N-terminal truncation and/or a C-terminal truncation of a reference polypeptide).

A “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least about 5, 10, 15, 20, 25, 30, 40, 50, 60, 70, 80, 90, 100, 150, 250, or 500 contiguous amino acid residues of a reference polypeptide. In some embodiments, a fragment may have a length within a range bounded by any value selected from 5, 10, 15, 20, 25, 30, 40, 50, 60, 70, 80, 90, 100, 150, 250, or 500, contiguous amino acid residues of a reference polypeptide. Fragments may be preferentially selected from certain regions of a molecule. The term “at least a fragment” encompasses the full length polypeptide.

A “full length” polynucleotide sequence is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polypeptide sequences. Homology, sequence similarity, and percentage sequence identity may be determined using methods in the art and described herein.

The phrases “percent identity” and “% identity,” for example, as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” may have substantially the same functional activity as a reference polypeptide.

Transient Interferon Blockade to Improve Immune Responses and Vaccines

Type I interferons (IFN-I) are critical for antiviral activity. Studies in the lymphocytic choriomeningitis virus infection (LCMV) model shown that IFN-I signaling improves the control of acute infection, but paradoxically, IFN-I signaling can also be immunosuppressive an impair the control of chronic infection. The role of IFN-I after vaccination, however, remains understudied. Vaccination is biologically distinct from natural infection. Vaccine-induced immune responses are typically elicited after in situ challenge with a low dose of an attenuated pathogen, resulting in a benign localized infection that induces immunological memory.

To evaluate the role of IFN-I during vaccination, we immunized C57BL/6 mice intramuscularly with vaccines expressing model antigens, including HIV antigens, co-administered with IFN-I blockers. IFN-I blockade resulted in significant improvement of T cell and B cell responses. Furthermore, IFN-I blockade also improved the immunogenicity of other viral vaccines, including rhabdovirus- flavivirus- and poxvirus based vaccines. Improvement of vaccine-elicited immunity was also observed using alternative immunization routes. Importantly, transient IFN-I blockade increased antigen availability at day 3 post-immunization, which resulted in improved antigen presentation and costimulation, and it did not prevent the clearance of the vaccine within a week of immunization, suggesting that the treatment could be safe. Our data suggest a model in which IFN-I transiently improves antigen availability at a critical time when the immune system is getting primed, resulting in long-lasting improvement of vaccine-induced immunity. Altogether, we show in multiple viral vector regimens, different routes, and different challenge models that transient IFN-I modulation improves vaccine-induced immunity. Therefore, acute modulation of IFN could be an effective strategy to improve vaccines.

As used herein, the term “IFN-I” refers the group of cytokines, including the more than ten forms of IFNα, IFNβ types 1 and 3, and several other related molecules that all bind to the same type 1 IFN receptor chain, IFNAR1.

As used herein, the term “IFNAR” refers to the IFN-I receptor that is comprised of two chains: IFNAR1 and IFNAR2.

The sequence of the mouse IFNAR1 is in the box below.

```
                10 20 30 40
MLAVVGAAAL VLVAGAPWVL PSAAGGENLK PPENIDVYII
                50 60 70 80
DDNYTLKWSS HGESMGSVTF SAEYRTKDEA KWLKVPECQH
                90 100 110 120
TTTTKCEFSL LDTNVYIKTQ FRVRAEEGNS TSSWNEVDPF
              130 140 150 160
IPFYTAHMSP PEVRLEAEDK AILVHISPPG QDGNMWALEK
              170 180 190 200
PSFSYTIRIW QKSSSDKKTI NSTYYVEKIP ELLPETTYCL
              210 220 230 240
EVKAIHPSLK KHSNYSTVQC ISTTVANKMP VPGNLQVDAQ
              250 260 270 280
GKSYVLKWDY IASADVLFRA QWLPGYSKSS SGSRSDKWKP
              290 300 310 320
IPTCANVQTT HCVFSQDTVY TGTFFLHVQA SEGNHTSFWS
              330 340 350 360
EEKFIDSQKH ILPPPPVITV TAMSDTLLVY VNCQDSTCDG
              370 380 390 400
LNYEIIFWEN TSNTKISMEK DGPEFTLKNL QPLTVYCVQA
              410 420 430 440
RVLFRALLNK TSNFSEKLCE KTRPGSFSTI WIITGLGVVF
              450 460 470 480
FSVMVLYALR SVWKYLCHVC FPPLKPPRSI DEFFSEPPSK
              490 500 510 520
NLVLLTAEEH TERCFIIENT DTVAVEVKHA PEEDLRKYSS
              530 540 550 560
QTSQDSGNYS NEEEESVGTE SGQAVLSKAP CGGPCSVPSP
              570 580 590
PGTLEDGTCF LGNEKYLQSP ALRTEPALLC
```

The sequence of the mouse IFNAR2 is in the box below:

```
                10 20 30 40
MRSRCTVSAV GLLSLCLVVS ASLETITPSA FDGYPDEPCT
                50 60 70 80
INITIRNSRL ILSWELENKS GPPANYTLWY TVMSKDENLT
                90 100 110 120
KVKNCSDTTK SSCDVTDKWL EGMESYVVAI VIVHRGDLTV
              130 140 150 160
CRCSDYIVPA NAPLEPPEFE IVGFTDHINV TMEFPPVTSK
              170 180 190 200
IIQEKMKTTP FVIKEQIGDS VRKKHEPKVN NVTGNFTFVL
              210 220 230 240
RDLLPKTNYC VSLYFDDDPA IKSPLKCIVL QPGQESGLSE
              250 260 270 280
SAIVGITTSC LVVMVFVSTI VMLKRIGYIC LKDNLPNVLN
              290 300 310 320
FRHFLTWIIP ERSPSEAIDR LEIIPTNKKK RLWNYDYEDG
              330 340 350 360
SDSDEEVPTA SVTGYTMHGL TGKPLQQTSD TSASPEDPLH
              370 380 390 400
EEDSGAEESD EAGAGAGAEP ELPTEAGAGP SEDPTGPYER
              410 420 430 440
RKSVLEDSFP REDNSSMDEP GDNIIFNVNL NSVFLRVLHD
              450 460 470 480
EDASETLSLE EDTILLDEGP QRTESDLRIA GGDRTQPPLP
              490 500 510
SLPSQDLWTE DGSSEKSDTS DSDADVGDGY IMR
```

The sequence of the human IFNAR1 is in the box below:

```
                10 20 30 40
MMVVILGATT LVLVAVAPWV LSAAAGGKNL KSPQKVEVDI
                50 60 70 80
IDDNFILRWN RSDESVGNVT FSFDYQKTGM DNWIKLSGCQ
                90 100 110 120
NITSTKCNFS SLKLNVYEEI KLRIRAEKEN TSSWYEVDSE
              130 140 150 160
TPFRKVENEL PPPENIEVSV QNQNYVLKWD YTYANMTFQV
              170 180
QWLHAFLKRN PGNHLYKWKQ IPDCENVKT
```

The sequence of the human IFNAR2 is in the box below:

```
                10 20 30 40
MLLSQNAFIF RSLNLVLMVY ISLVFGISYD SPDYTDESCT
                50 60 70 80
FKISLRNFRS ILSWELKNHS IVPTHYTLLY TIMSKPEDLK
                90 100 110 120
VVKNCANTTR SFCDLTDEWR STHEAYVTVL EGFSGNTTLF
              130 140 150 160
SCSHNFWLAI DMSFEPPEFE IVGFTNHINV MVKFPSIVEE
              170 180 190 200
ELQFDLSLVI EEQSEGIVKK HKPEIKGNMS GNFTYIIDKL
              210 220 230 240
IPNTNYCVSV YLEHSDEQAV IKSPLKCTLL PPGQESESAE
              250 260 270 280
SAKIGGIITV FLIALVLTST IVTLKWIGYI CLRNSLPKVL
              290 300 310 320
NFHNFLAWPF PNLPPLEAMD MVEVIYINRK KKVWDYNYDD
              330 340 350 360
ESDSDTEAAP RTSGGGYTMH GLTVRPLGQA SATSTESQLI
              370 380 390 400
DPESEEEPDL PEVDVELPTM PKDSPQQLEL LSGPCERRKS
              410 420 430 440
PLQDPFPEED YSSTEGSGGR ITFNVDLNSV FLRVLDDEDS
              450 460 470 480
DDLEAPLMLS SHLEEMVDPE DPDNVQSNHL LASGEGTQPT
              490 500 510
FPSPSSEGLW SEDAPSDQSD TSESDVDLGD GYIMR
```

As used herein, the term “IFN-I blocking agent,” or “IFN-I blocker” referrers to compounds that directly or indirectly inhibit or attenuate the effect of IFN-I binding to the IFNANF1R1. In some embodiments, the IFN-I blocker is an antibody that binds an IFN-I. In some embodiments, the IFN-I blocker is an antibody that binds the IFN-I receptor. For example, in some embodiments, the INF-I blocking agent binds to the IFN receptor chain IFNAR2. In some embodiments, the INF-I blocking agent binds to the IFN receptor chain IFNAR1. In some embodiments, the INF-I blocking agent binds to the IFNAR1 at the SD3 subdomain, as described for example, in Weerd, et al., “A hot-spot on Interferon alpha/beta receptor subunit 1 (IFNAR1) underpins its interaction with Interferon-beta and dictates signaling,” JBC Papers in Press. Published on Mar. 13, 2017 as manuscript M116.773788 at http://www/jbc.org/cgi/doi/10.1074/jbc.M116.773788, herein incorporated by reference in its entirety. In some embodiments, the IFN-I blocking agent binds residue R279 of the SD3 subdomain, for example as described in Peng et al., “Molecular basis for antagonistic activity of anifrolumab, an anti-interferon-alpha receptor 1 antibody,” mAbs 7:2, 428-439; March/April 2015, available at https://www.ncbi.nlm.nih.gov/pmc/articles/PMC4622752/, herein incorporated by reference in its entirety.

An exemplary IFN receptor blocking antibody is anifrolumab, which has shown acceptable safety profiles in prior clinical trials in humans with lupus erythematosus. Since anti-IFN receptor antibodies have already undergone clinical testing in humans, and since it has good safety profiles, such compounds would be useful in methods and compositions of the present invention. Another exemplary IFN-1 blocker is αIFNAR1 clone MAR1-5A3, which is commercially available. Method of making antibodies that bind IFN-I and IFNAR1 are well known in the art, and exemplary antibodies are commercially available. Any antibody that prevents an IFN-I molecule from binding to its receptor is an IFN-I blocker, as used herein. For example, an antibody that includes the CDRs of anifrolumab, and/or the CDRs of MAR1-5A3 and functions to block IFN-I receptor function, i.e., precluding binding of IFN-I isoforms, and thus preventing the upregulation of interferon stimulated genes (ISG) is an IFN-1 blocking agent as defined herein.

As used herein, the term “enhanced vaccine efficacy” refers to improved immune response, vaccine immunogenicity, and/or enhanced immune protection following a vaccine administration and follow-up antigen challenge in a subject, as compared to a control subject (e.g., a subject who does not receive an IFN-I blocker). By way of example, but not by way of limitation, such enhancements include, but are not limited to 1) an increase in CD8 T-cell response; 2) an increase in CD4 T-cells; 3) an increase in antibodies; 4) an increase in heterologous antibodies; 5) a lack of, or reduction in, disease symptoms; 6) a lack of or reduction in the antigen in the subject compared to a control subject after challenge (e.g., a lower viral load). As used herein, “potentiating” or “enhancing” an immune response also refers to increasing the magnitude and/or the breadth of the immune response. For example, the number of immune cells that recognize a particular epitope may be increased (“magnitude”) and/or the numbers of epitopes that are recognized may be increased (“breadth”). Preferably, a 2-fold, a 5-fold, or in some embodiments, a 10-fold or greater, enhancement in CD8 and/or CD4 T-cell responses may be obtained by administering the pharmaceutical composition disclosed herein.

As used herein, a “CD8 response” is referred to as the ability of cytotoxic CD8 T-cells to recognize and kill cells expressing foreign peptides in the context of a major histocompatibility complex (MHC) class I molecule. These cells are important for controlling infections and cancers.

As used herein, “viral load” is the amount of virus present in the blood of a patient or animal. Viral load is also referred to as viral titer or viremia. Viral load can be measured in variety of standard ways including by plaque assays or copy Equivalents of the viral RNA (vRNA) genome per milliliter blood plasma (vRNA copy Eq/ml). This quantity may be determined by standard methods that include RT-PCR. In some embodiments, the composition disclosed herein (compositions including IFN-I blockers), after being administered to a subject in need thereof, result in a reduction in the viral load (upon subsequent challenge) in said subject compared to a control subject that did not previously receive and IFN-I blocker at the time of immunization.

As used herein the term “vaccine” refers to a substance used to generate immune responses, including antibodies, and provide immunity against one or several diseases from the causative agent of a disease, its products, or a synthetic substitute, treated to act as an antigen without inducing the disease. In some embodiments, the vaccine is a viral vaccine (i.e., a vaccine to ameliorate or prevent a disease caused by a natural infection). Numerous viral vaccines useful in the present methods and compositions, are commercially available and licensed for use in humans and other animals, and are well known in the art. Exemplary viral vaccines that may be improved by this invention include but are not limited to influenza vaccines, hepatitis A and B vaccines, human papilloma virus vaccine, zoster vaccine, smallpox vaccine, measles vaccine, rabies vaccine, poliovirus vaccine, Japanese encephalitis vaccine, rubella vaccine, rotavirus vaccine, yellow fever vaccine, varicella virus vaccine, lassa/machupo/junin/guanarito (and other hemorrhagic arenaviruses) vaccines, ebola virus vaccine, HIV vaccine, and cancer vaccine. Many other live attenuated viral vaccines may be improved by transient IFN-I modulation.

As used herein “HIV” refers to “human immunodeficiency virus” which may include human immunodeficiency virus type 1 (i.e., “HIV-1”) and human immunodeficiency virus type 2 (i.e., “HIV-2”).

Exemplary Applications

IFN-I blockers of the present disclosure are useful to improve immune responses against antigens, and are useful to improve the efficacy of HIV vaccines, cancer vaccines, and other experimental vaccines. Additionally, an IFN-I blocker as disclosed herein is also useful to improve the efficacy of already licensed vaccines. IFN-I blockade could also be used to improve the efficacy/generation of monoclonal antibodies. Because the IFN-I blockade as disclosed herein improves antibody response after vaccination or natural infection, it can also be used to increase the generation of new and more potent monoclonal antibodies for combatting infectious disease or cancer.

Pharmaceutical Compositions

The disclosed IFN-I blocking agents and/or antigens may be formulated as pharmaceutical compositions for administering to a subject in need thereof, for examples, such as vaccines. The disclosed pharmaceutical compositions may include: (a) a vaccine; (b) an IFN-I blocking agent; and (c) one or more pharmaceutically acceptable adjuvants, carriers, excipients, or diluents. Alternatively, a pharmaceutical composition may include (a) an IFN-I blocking agent; and (b) one or more pharmaceutically acceptable carriers, excipients, or diluents.

The IFN-I blocking agents utilized in the methods disclosed herein may be formulated as a pharmaceutical composition that includes one or more carrier agents, adjuvants, binding agents, filling agents, lubricating agents, suspending agents, buffers, wetting agents, and/or preservatives. Examples of preservatives may include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

The disclosed methods and compositions may utilize and/or comprise adjuvants. Examples of adjuvants which may be employed in the present compositions and methods include, but are not limited to MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TLR4 ligand, a monophosphoryl lipid A, together in a lipid or liposomal carrier. Other adjuvants include, but are not limited to, nonionic block co-polymer adjuvants (e.g., CRL1005), aluminum phosphates (e.g., AIPO.sub.4), R-848 (a Th1-like adjuvant), imiquimod, PAM3CYS, poly (I:C), loxoribine, potentially useful human adjuvants such as BCG (bacille Calmette-Guerin) and Corynebacterium parvum, CpG oligodeoxynucleotides (ODN), cholera toxin derived antigens (e.g., CTA1-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

The IFN-I blocking agents utilized in the methods disclosed herein may be formulated as a pharmaceutical composition for delivery via any suitable route for eliciting an immune response. The therapeutic agents utilized in the methods disclosed herein may be administered in conventional dosage forms prepared by combining the active ingredient with standard pharmaceutical carriers or diluents according to conventional procedures well known in the art. These procedures may involve mixing or dissolving the ingredients as appropriate to the desired preparation.

Pharmaceutical compositions comprising the therapeutic agents may be adapted for administration by any appropriate route, by way of example, but not by way of limitation, topically, parenterally, orally, nasally, or by inhalation. Typical delivery routes include parenteral administration (e.g., intradermal, intramuscular, subcutaneous, intravenous delivery, e.g., injectable administration). Such formulations may be prepared by any method known in the art of pharmacy, for example by bringing into association the active ingredient with the carrier(s) or excipient(s).

Compositions disclosed herein can be formulated and/or administered in dosages and by techniques well known to those skilled in the medical arts taking into consideration such factors as the age, sex, weight, and condition of the particular patient, and the route of administration.

The pharmaceutical compositions may be administered prophylactically or therapeutically. In prophylactic administration, the compositions (e.g., a vaccine and an IFN-I blocking agent) may be administered in an amount sufficient to induce CD8+, CD4+, and/or antibody responses for protecting against infection. In therapeutic applications, the vaccines are administered to a patient in an amount sufficient to elicit a therapeutic effect (e.g., a CD8+, CD4+, and/or antibody responses against an antigen associated with an infectious agent (e.g., a virus) or a disease (e.g., cancer), which cures or at least partially arrests or slows symptoms and/or complications of the infection or disease (i.e., a “therapeutically effective dose” or a “therapeutically effective amount”).

A vaccine may be co-administered with the IFN-I blocking agent, or the IFN-I blocking agent may be administered sequentially, either before or after, vaccine administration. Preferably, the vaccine and the IFN-I blocking agent are co-administered, or are administered to the subject in the same day. In some embodiments, the vaccine is administered to the subject and the IFN-I blocking agent is administered separately, either before or after the vaccine, but within 24 hours (1 day), within 48 hours (2 days), within 72 hours (3 days), within 96 hours (4 days), within 120 hours (5 days), or within one week of vaccine administration.

### Illustrative Embodiments

The following Embodiments are illustrative and should not be interpreted to limit the scope of the claimed subject matter.

Embodiment 1. A method for enhancing an immune response in a subject in need thereof, the method comprising administering a pharmaceutical composition comprising an effective amount of a therapeutic agent that blocks the activity of interferon and a pharmaceutical composition comprising an effective amount of an antigen.

Embodiment 2. The method of embodiment 1, wherein the method vaccinates the subject against an infectious agent (e.g., a virus or a bacteria) or disease (e.g., cancer) that comprises the antigen.

Embodiment 3. The method of embodiment 1 or 2, wherein the pharmaceutical composition comprising the effective amount of the antigen is a viral vaccine.

Embodiment 4. The method of embodiment 3, wherein the viral vaccine is selected from the group consisting of an influenza vaccine, a hepatitis A vaccine, a hepatitis B vaccine, a human papilloma virus vaccine, a zoster vaccine, a smallpox vaccine, a measles vaccine, a rabies vaccine, a poliovirus vaccine, a Japanese encephalifis vaccine, a rubella vaccine, a rotavirus vaccine, a yellow fever vaccine, a varicella virus vaccine, an HIV vaccine, and any combination thereof.

Embodiment 5. The method of embodiment 1 or 2, wherein the pharmaceutical composition comprising the effective amount of the antigen is an anti-cancer vaccine.

Embodiment 6. The method of any one of embodiments 1-5, wherein the therapeutic agent that blocks the activity of IFN-I comprises an antibody.

Embodiment 7. The method of any of embodiments 1-5, wherein the therapeutic agent that blocks the activity of IFN-I comprises an IFN-I receptor binding antibody.

Embodiment 8. The method of any of embodiments 1-5, wherein the therapeutic agent that blocks the activity of IFN-I comprises αIFNAR1 clone MAR1-5A3.

Embodiment 9. The method of any of embodiments 1-5, wherein the therapeutic agent that blocks the activity of IFN-I comprises anifrolumab.

Embodiment 10. The method of any one of embodiments 1-9, wherein the pharmaceutical composition comprising the effective amount of the therapeutic agent that blocks the activity of the IFN-I pathway is admixed with the pharmaceutical composition comprising the effective amount of the antigen prior to administering to the subject.

Embodiment 11. The method of any one of embodiments 1-8, wherein the pharmaceutical composition comprising the effective amount of the therapeutic agent that blocks the activity of IFN-I is administered separately from the pharmaceutical composition comprising the effective amount of the antigen.

Embodiment 12. The method of any one of embodiments 1-9 and 11, wherein the pharmaceutical composition comprising the effective amount of the therapeutic agent that blocks the activity of IFN-I is administered within 24 hours, within 48 hours, or within 72 hours of administering the pharmaceutical composition comprising the effective amount of the antigen.

Embodiment 13. The method of any one of embodiments 1-12, wherein the improved immune response comprises one or more of the following, optionally after the subject further subsequently is administered the antigen and/or the subject is later challenged with an infectious agent (e.g. a virus or a bacteria) or disease (e.g., cancer) comprising the antigen: improved vaccine immunogenicity and/or enhanced immune protection; an increase in antigen-specific CD8 T-cells; an increase in antigen-specific CD4 T-cells; an increase in antigen-specific B cells or antibodies; an increase in heterologous antibodies; a lack of, or reduction in, disease symptoms; and/or a lack of, or reduction in the antigen.

Embodiment 14. An immunogenic composition comprising: an effective amount of an antigen; and a therapeutic agent that blocks the activity of IFN-1.

Embodiment 15. The immunogenic composition of embodiment 14, wherein the immunogenic composition comprises a vaccine against an infectious agent that comprises the antigen.

Embodiment 16. The composition of embodiment 14 or 15, wherein the composition comprises a vaccine selected from the group consisting of: an influenza vaccine, a hepatitis A vaccine, a hepatitis B vaccine, a human papilloma virus vaccine, a zoster vaccine, a smallpox vaccine, a measles vaccine, a rabies vaccine, a poliovirus vaccine, a Japanese encephalitis vaccine, a rubella vaccine, a rotavirus vaccine, a yellow fever vaccine, a varicella virus vaccine, an HIV vaccine, and other replicating viral vaccines, and combinations thereof.

Embodiment 17. The composition of embodiment 14, wherein the composition comprises an anti-cancer vaccine.

Embodiment 18. The composition of any of embodiments 14-17, wherein the therapeutic agent that blocks the activity of IFN-I comprises an antibody.

Embodiment 19. The composition of any of embodiments 14-17, wherein the therapeutic agent that blocks the activity of IFN-I comprises an IFN-I receptor binding antibody.

Embodiment 20. The composition of any of embodiments 14-17, wherein the therapeutic agent that blocks the activity of IFN-I comprises αIFNAR1 clone MAR1-5A3.

Embodiment 21. The composition of any of embodiments 14-17, wherein the therapeutic agent that blocks the activity of IFN-I comprises anifrolumab.

Embodiment 22. A kit comprising: (a) an antigen, optionally wherein the kit comprises a vaccine; and (b) an IFN-I blocking agent.

Embodiment 23. The kit of embodiment 22, wherein the kit comprises a viral vaccine, and wherein the IFN-I blocking agent comprises an IFN-I receptor binding antibody.

Embodiment 24. The kit of embodiment 22, wherein the kit comprises an anti-cancer vaccine, and wherein the IFN-I blocking agent comprises an IFN-I receptor binding antibody.

### EXAMPLES

The following Examples are illustrative and should not be interpreted to limit the scope of the claimed subject matter.

Transient IFN-I Blockade Improves Vaccine Efficacy

Abstract

Type I interferons (IFN-I) are a major antiviral defense and are critical for the activation of the adaptive immune system. However, early viral clearance by IFN-I could limit antigen availability, which could in turn impinge upon the priming of the adaptive immune system. In this study, we hypothesized that transient IFN-I blockade could increase antigen presentation after acute viral infection. To test this hypothesis, we infected mice with viruses co-administered with a single dose of IFN-I receptor-blocking antibody to induce a short-term blockade of the IFN-I pathway. This resulted in a transient “spike” in antigen levels, followed by rapid antigen clearance. Interestingly, short-term IFN-I blockade after coronavirus, flavivirus, rhabdovirus, or arenavirus infection induced a long-lasting enhancement of immunological memory that conferred improved protection upon subsequent reinfections. Short-term IFN-I blockade also improved the efficacy of viral vaccines. These findings demonstrate a novel mechanism by which IFN-I regulate immunological memory and provide insights for rational vaccine design.

Introduction

Type I interferons (IFN-I) are a first line of defense during viral infection. Absence of IFN-I results in disseminated viral infections and impaired priming of adaptive immune responses (Burns et al., 2016; Ciancanelli et al., 2015; Duncan et al., 2015; Dupuis et al., 2003; Erickson and Pfeiffer, 2013; Hambleton et al., 2013; Hernandez et al., 2019; Hernandez et al., 2018; Hoyos-Bachiloglu et al., 2017; Kolumam et al., 2005; Kreins et al., 2015; Le Bon et al., 2003; Minegishi et al., 2006; Moens et al., 2017; Sandler et al., 2014; Shahni et al., 2015; Stark et al., 1998; Teijaro et al., 2013; Wilson et al., 2013). Robust IFN-I responses are also a hallmark of effective vaccines (Gaucher et al., 2008; Li et al., 2017; Pulendran and Ahmed, 2011; Pulendran et al., 2010; Querec et al., 2009). However, there is an overlooked paradox between IFN-I and the elicitation of long-lived antiviral immunity: IFN-I can restrict initial viral replication, which in turn could limit antigen availability at a critical time when the adaptive immune system is getting primed. This conundrum motivated us to analyze the effects of transiently blocking IFN-I early after viral infection, with the goal of augmenting hyperacute antigen levels and subsequent antigen priming. Here, we show that short-term IFN-I blockade during an initial viral infection results in a profound improvement of immunological memory, rendering the host better protected against subsequent reinfections with similar or more virulent pathogens. These findings are important for two reasons. First, they highlight an interesting mechanism by which innate immunity regulates long-lived immunological memory. Second, they may have important implications for rational vaccine design.

Results

Short-Term IFN-I Blockade During Acute Viral Infection Improves Immunological Memory

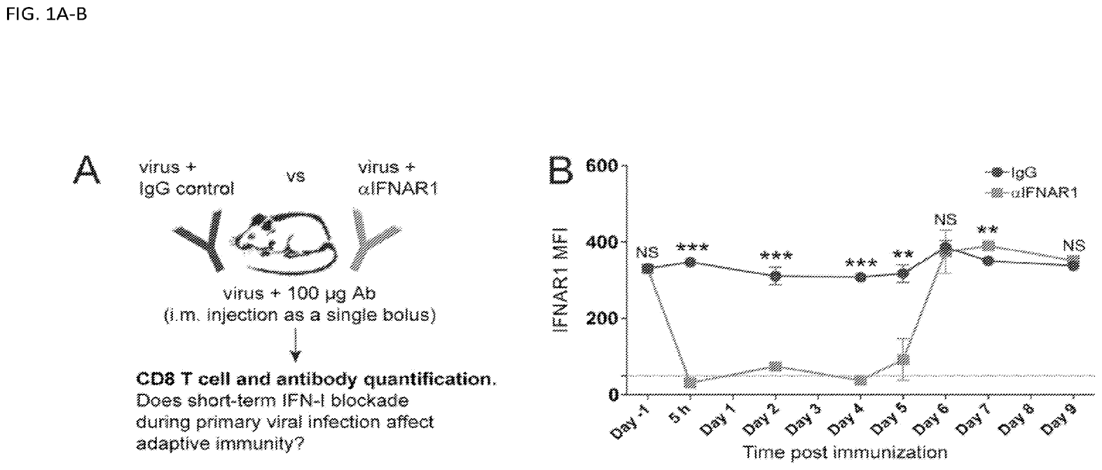

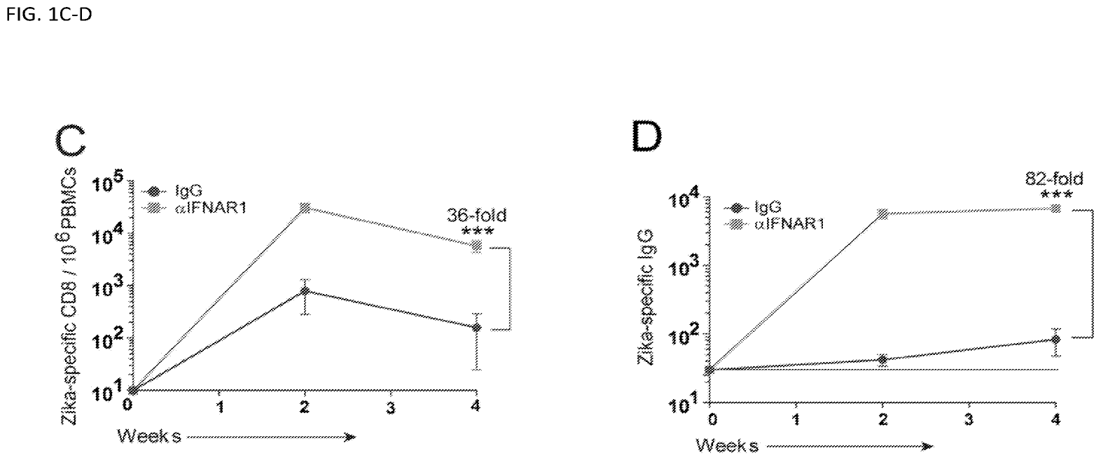

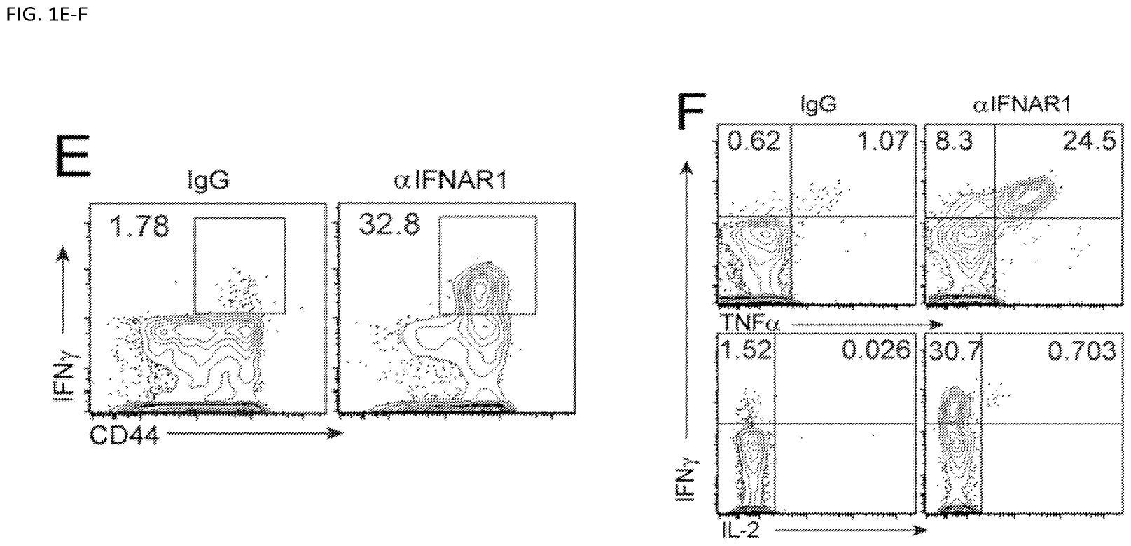

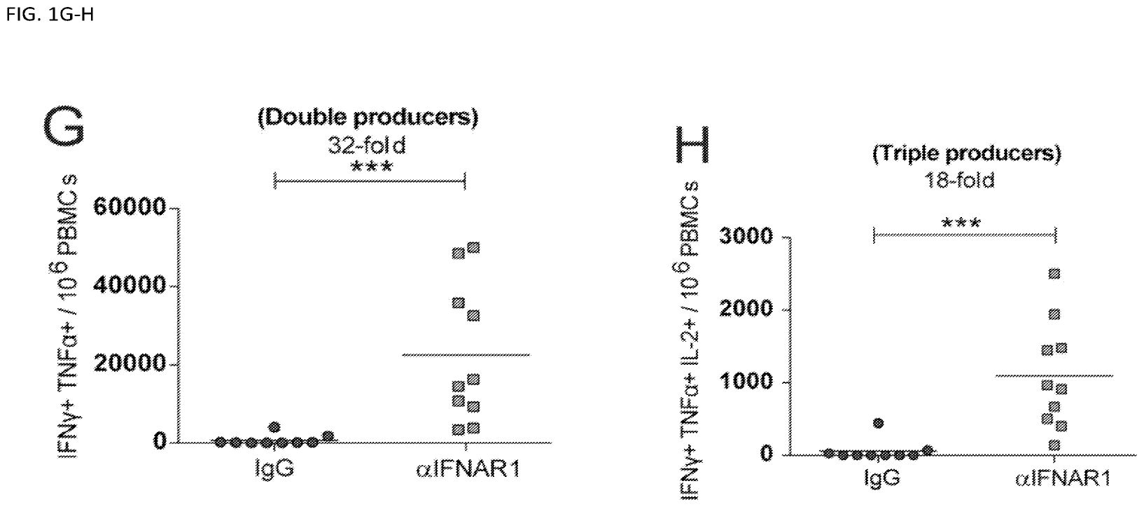

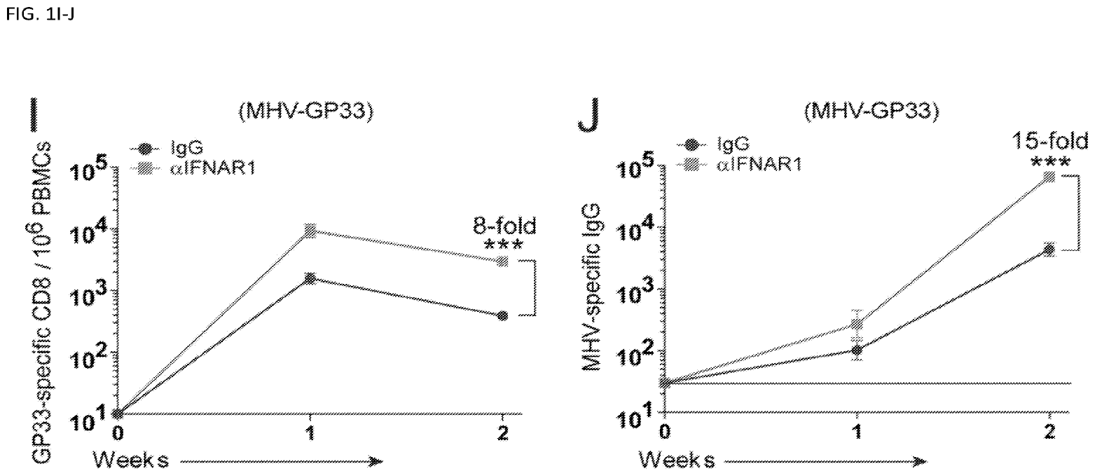

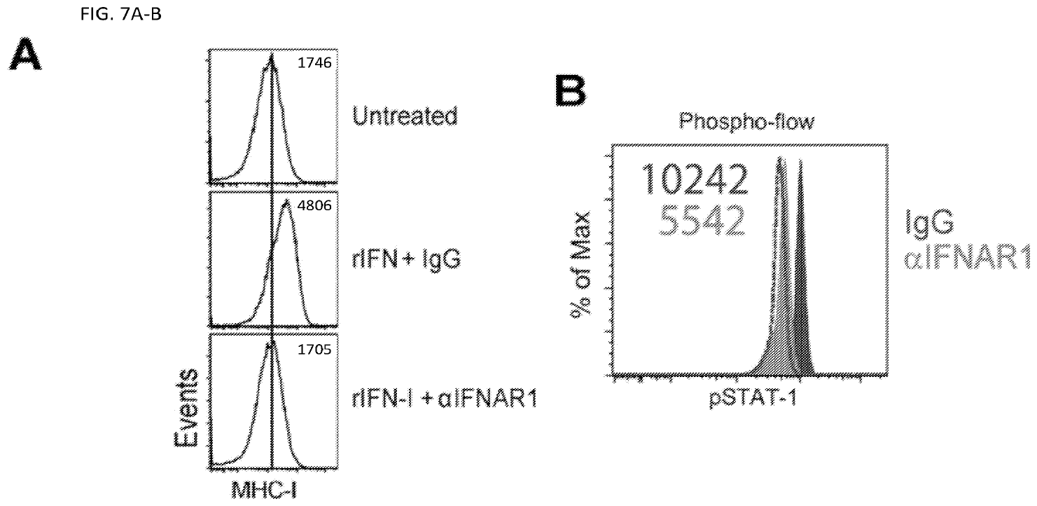

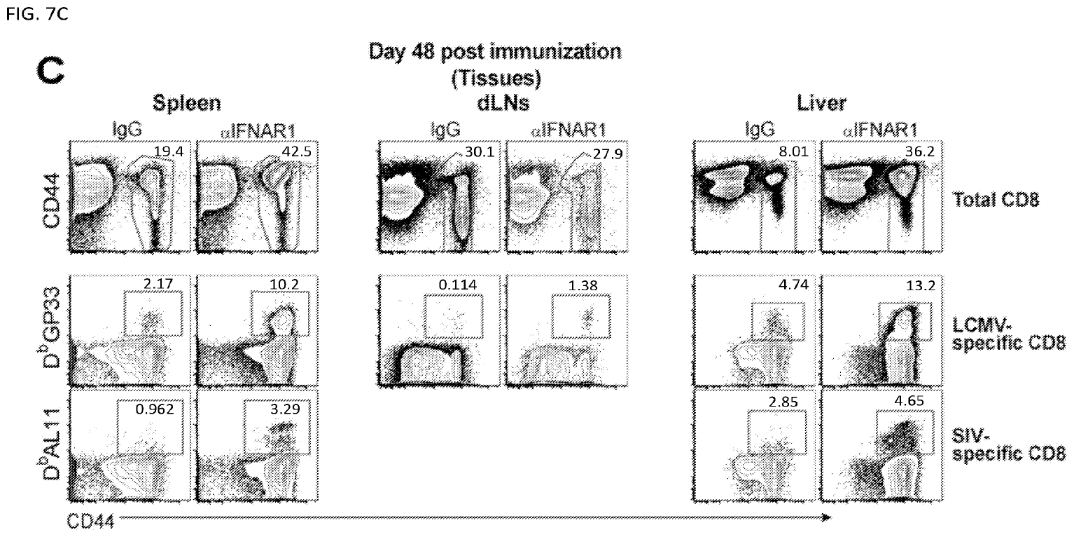

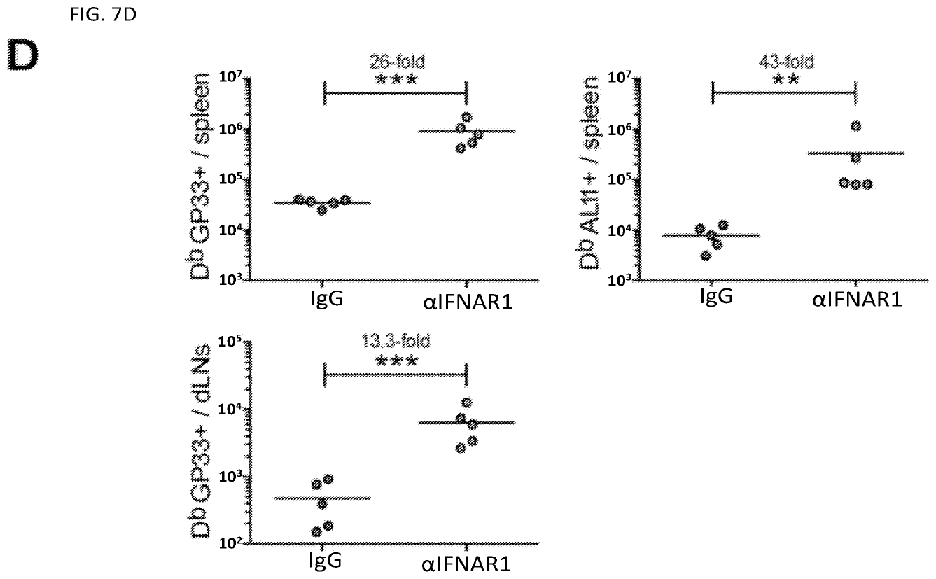

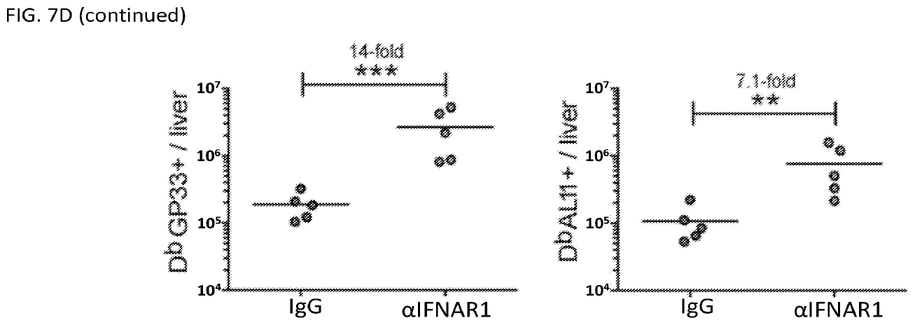

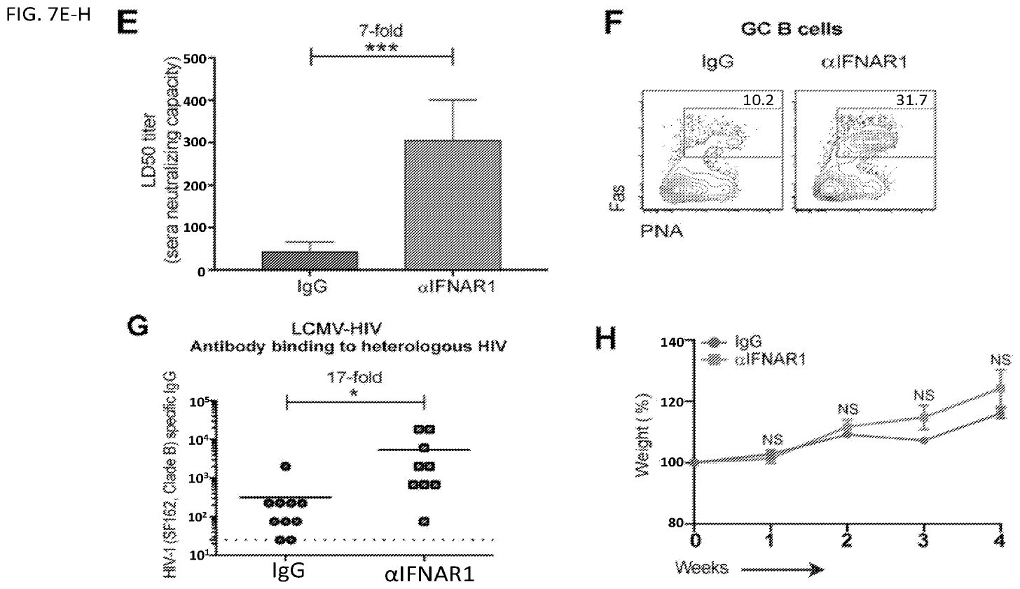

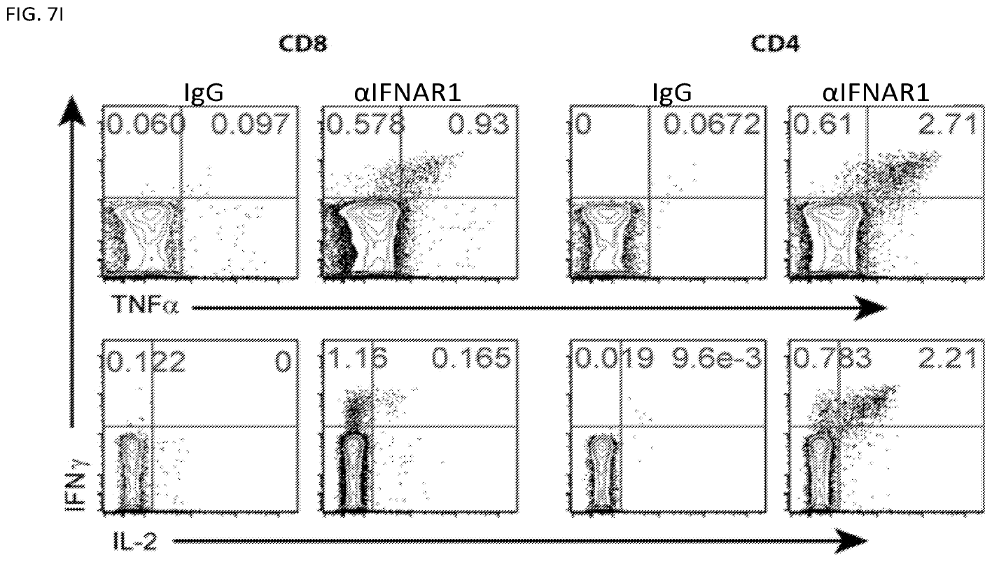

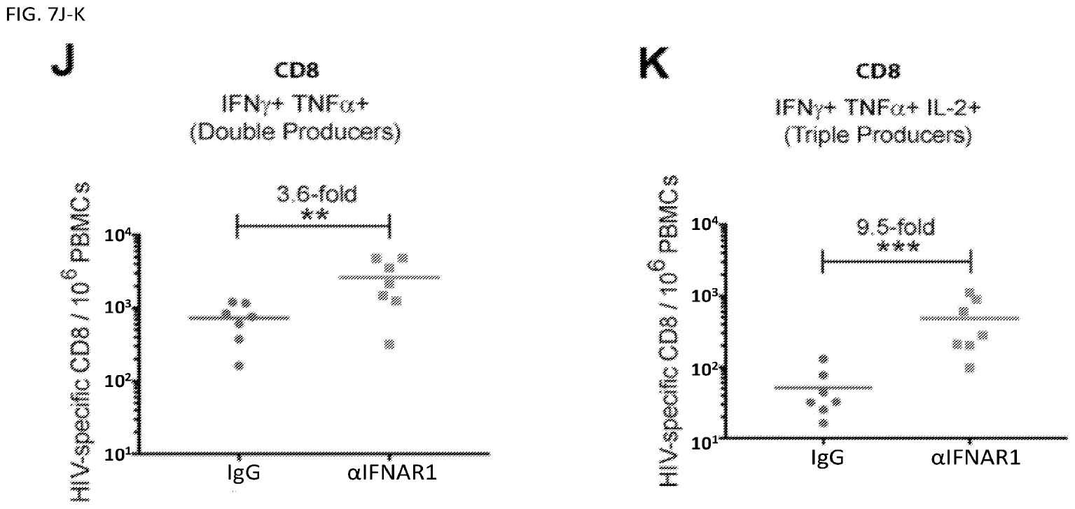

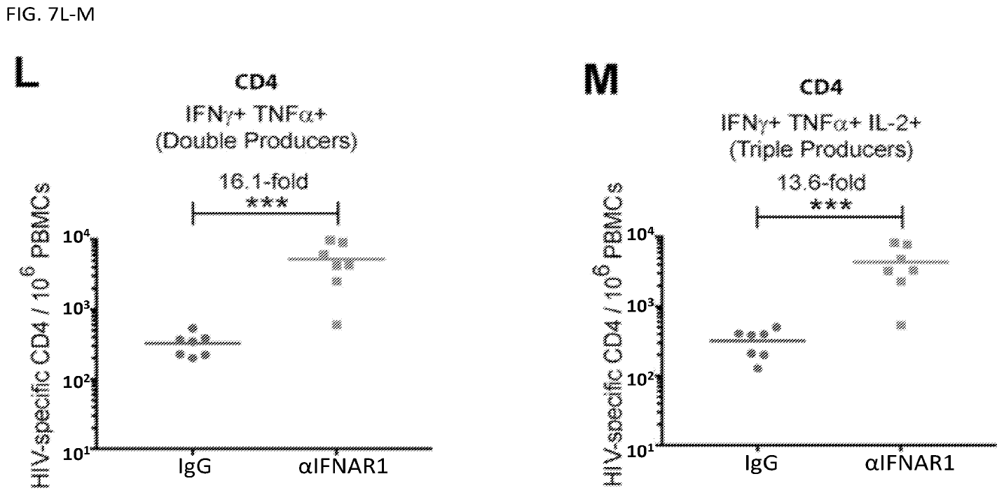

Discovered more than six decades ago, IFN-I have been shown to play an indispensable role in antiviral immunity. Long-term defects in the IFN-I pathway result in impairment of immune responses following acute viral infections or vaccinations. However, it is currently unclear whether a short-term blockade of IFN-I would have a similar effect. To transiently block the IFN-I pathway, we used an IFN-I receptor-blocking antibody (αIFNAR1, clone MAR1-5A3) that has been used in prior studies (Bhattacharyya et al., 2017; Teijaro et al., 2013; Wang et al., 2019; Wilson et al., 2013). We first corroborated that this antibody blocks IFN-I signaling in vitro (FIG. 7A). We then immunized mice with different viruses, which were coadministered with control IgG or αIFNAR1 to induce a short-term blockade of the IFN-I pathway (FIG. 1A). This single dose of αIFNAR1 resulted in reduced STAT1 phosphorylation (FIG. 7B) and a short-term blockade of the IFN-I receptor lasting 96 h (FIG. 1B). In these experiments, we co-administered the αIFNAR1 antibody locally (intramuscularly) together with the virus. As shown in FIG. 1B, a fraction of the antibody goes systemic, since there is blockade of IFNAR1 in the blood. Interestingly, short-term IFN-I blockade after a Zika virus infection resulted in a 36-fold improvement in CD8 T cell responses and an 82-fold improvement in antibody responses (FIG. 1, C-E). Short-term IFN-I blockade also resulted in a substantial increase in cytokine co-expressing T cells relative to control immunized mice (FIG. 1, F-H). Short-term IFN-I blockade also improved adaptive immune responses following an acute coronavirus infection with mouse hepatitis virus (MHV; FIG. 1, I and J), which is in the same genera as SARS-CoV (Betacoronaviridae). These data showed that short-term or hyperacute blockade of IFN-I improves the immunogenicity of acute viral infections. We then evaluated whether these same effects could apply to viral vaccines.

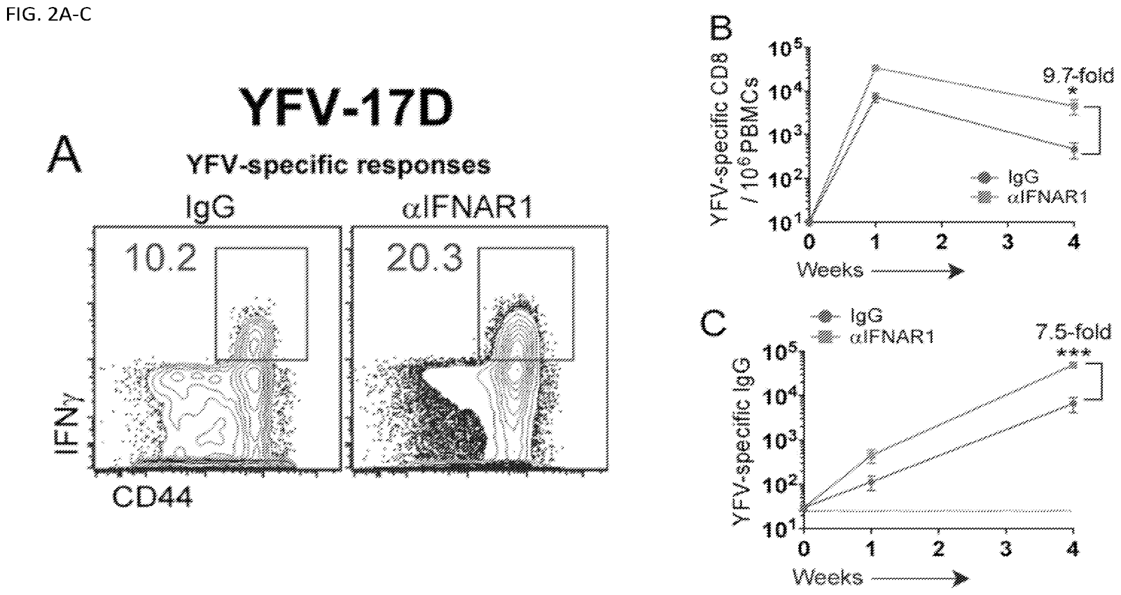

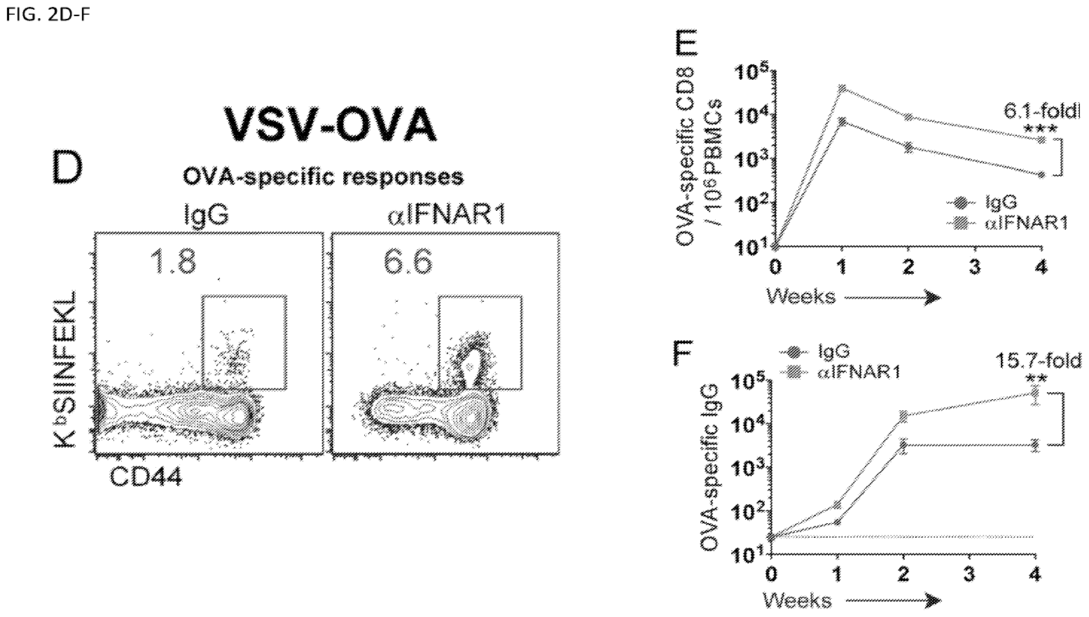

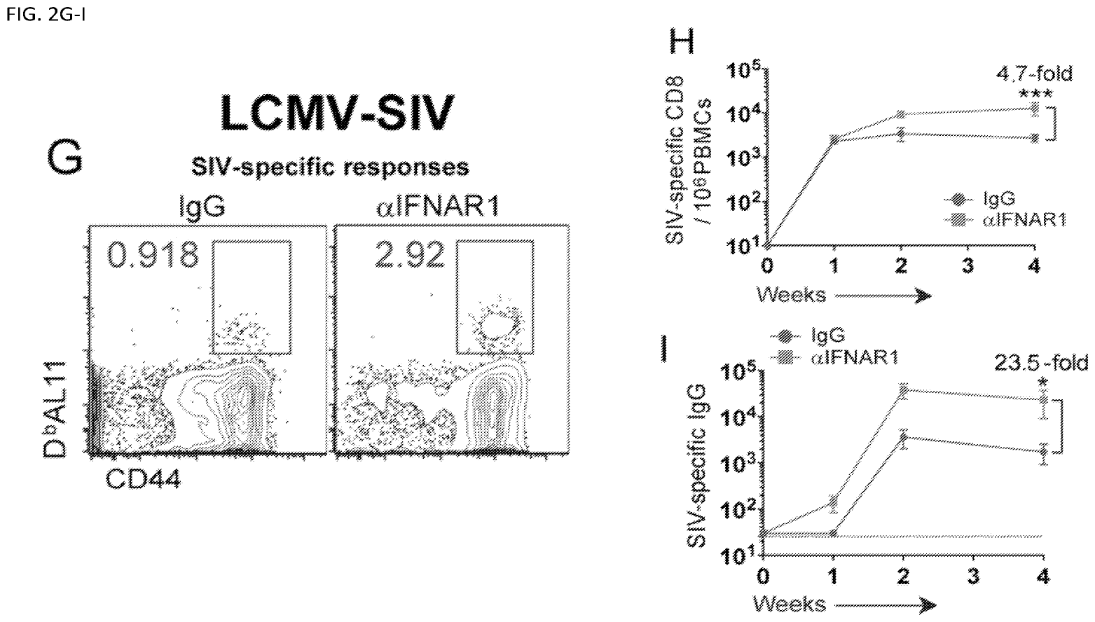

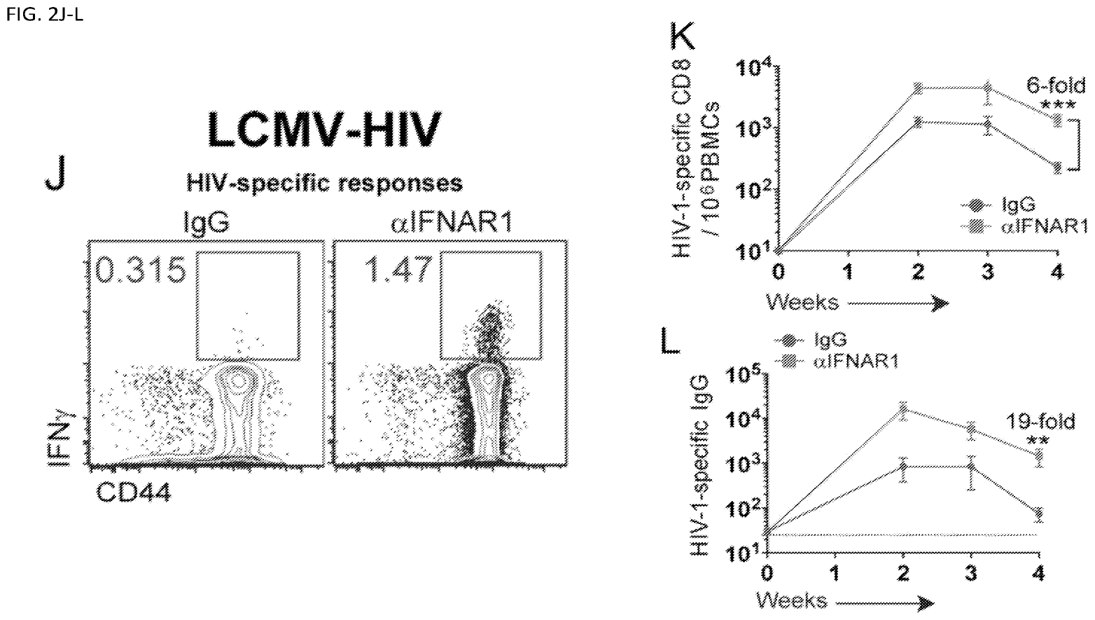

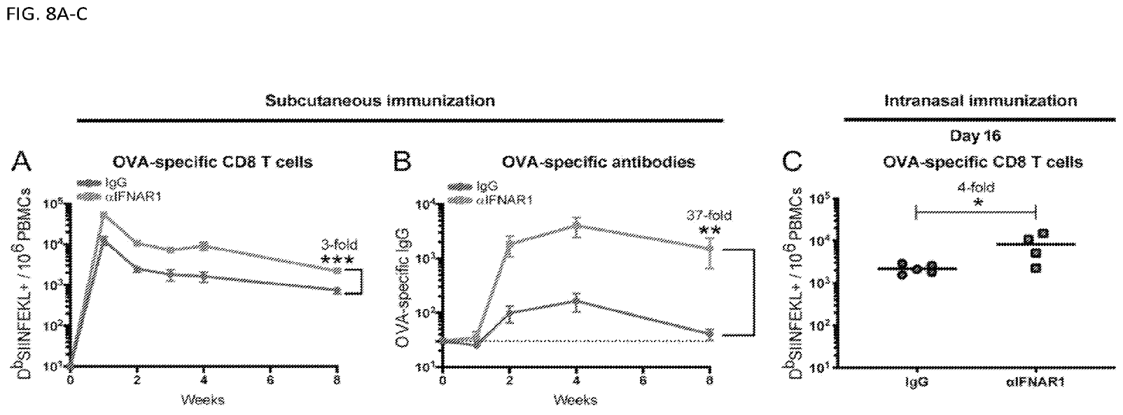

Interestingly, IFN-I blockade improved the immunogenicity of the clinically approved yellow fever virus vaccine (YFV-17D; FIG. 2, A-C). Rhabdovirus- and arena-virus based vaccines were also significantly improved after short term IFN-I blockade (FIG. 2, D-L; and FIG. 7, C and D). IFN-I blockade during lymphocytic choriomeningitis virus (LCMV)—simian immunodeficiency virus (SIV) vaccination significantly increased antibody-mediated SIV neutralization compared with control, as measured by in vitro neutralization assays (FIG. 7E). In the context of LCMV-HIV vaccination, short-term IFN-I blockade facilitated germinal center B cell responses (FIG. 7F), which suggested that antibody diversification was improved. A main challenge for developing vaccines against highly evolving viruses, such as HIV, is that vaccines based on a specific viral strain may not confer substantial cross-reactive humoral immunity against heterologous viral strains. However, IFN-I blockade resulted in a 17-fold improvement in cross-reactive humoral immunity after vaccination (FIG. 7G). Overall, our short-term IFN-I blockade regimen seemed well tolerated, and mice did not exhibit overt weight loss after infection (FIG. 7H). In addition, IFN-I blockade improved cytokine co-expression by HIV specific T cells compared with control vaccination (FIG. 7, I-M). Altogether, these data with different viral vaccines suggest that short-term IFN-I blockade paradoxically acts as a potent adjuvant. The data above involved intramuscular infection/vaccination, so we investigated whether these effects were dependent on the route. We observed a similar increase in immune responses when mice were injected via the subcutaneous or intranasal routes (FIG. 8), suggesting that our observations were not dependent on the route of infection.

Transcriptional and Virological Analyses

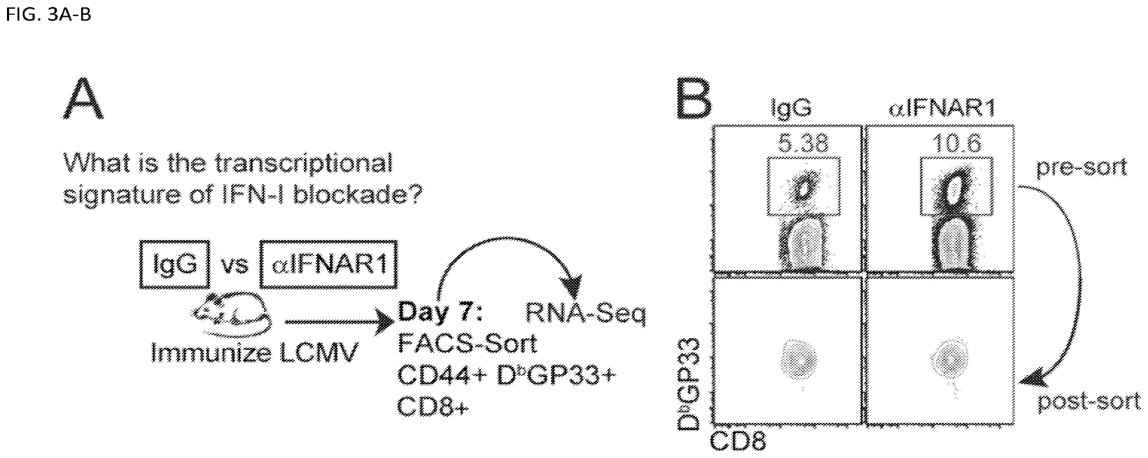

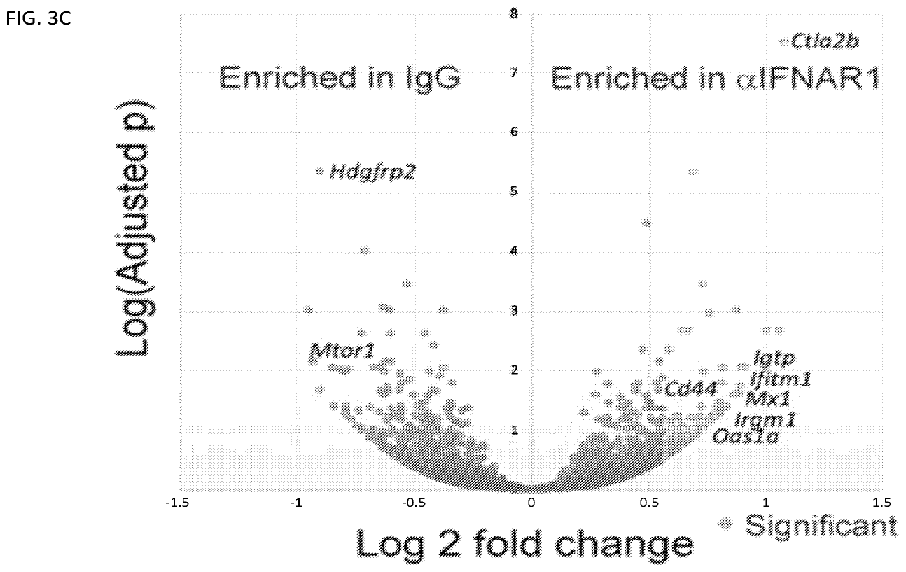

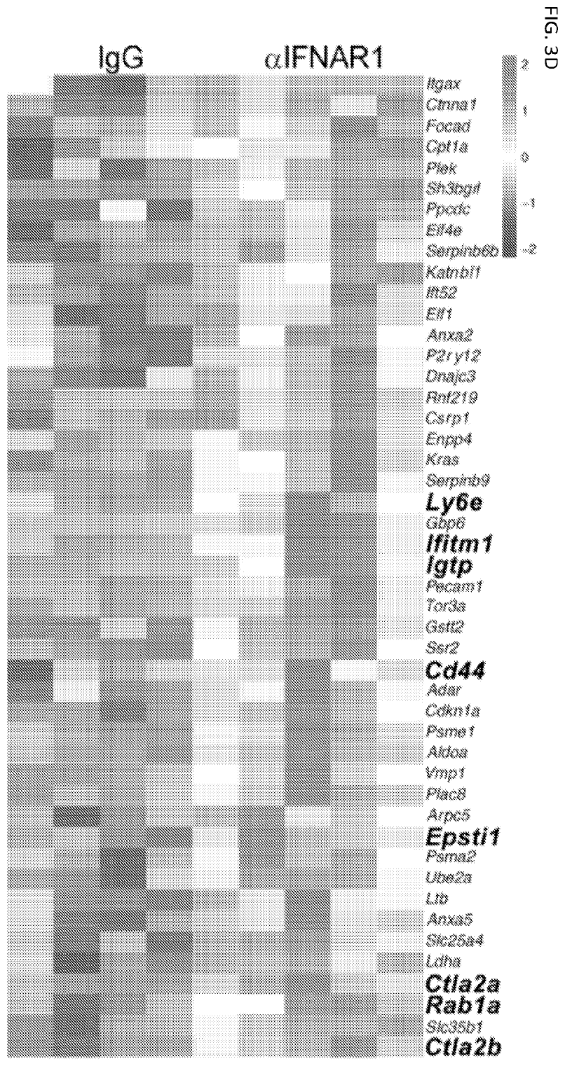

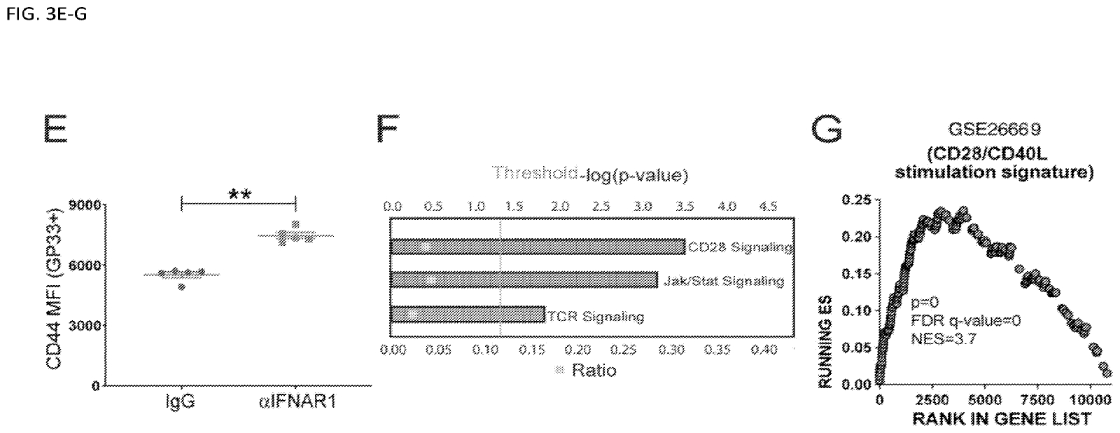

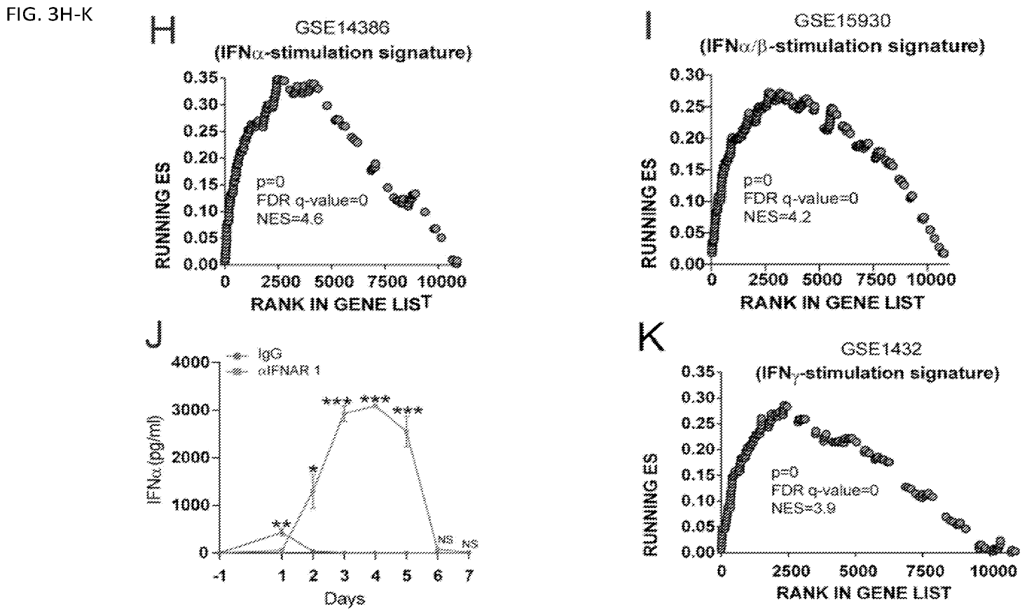

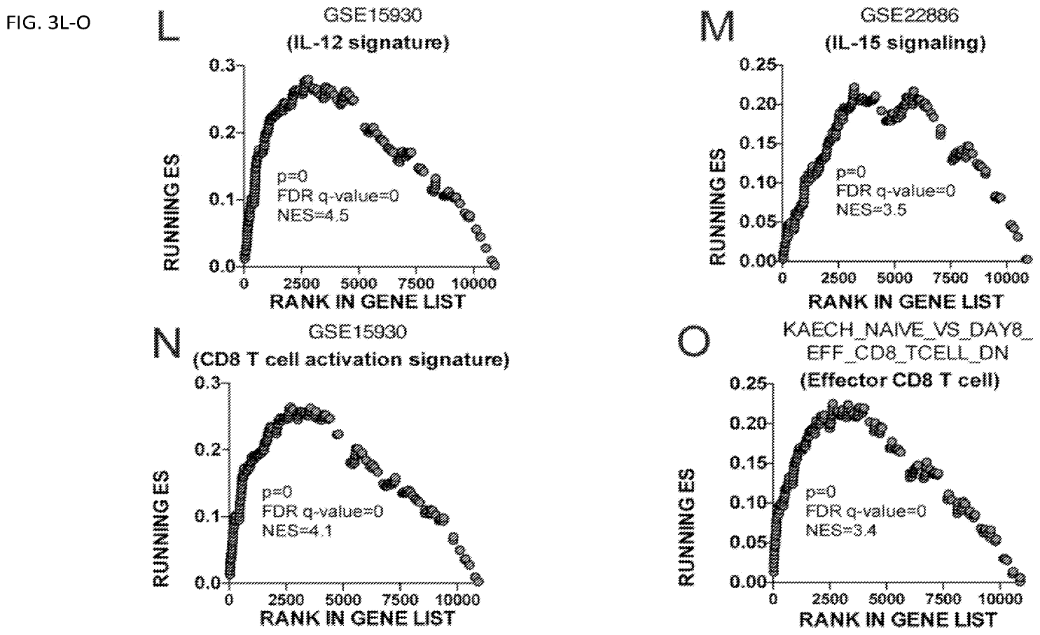

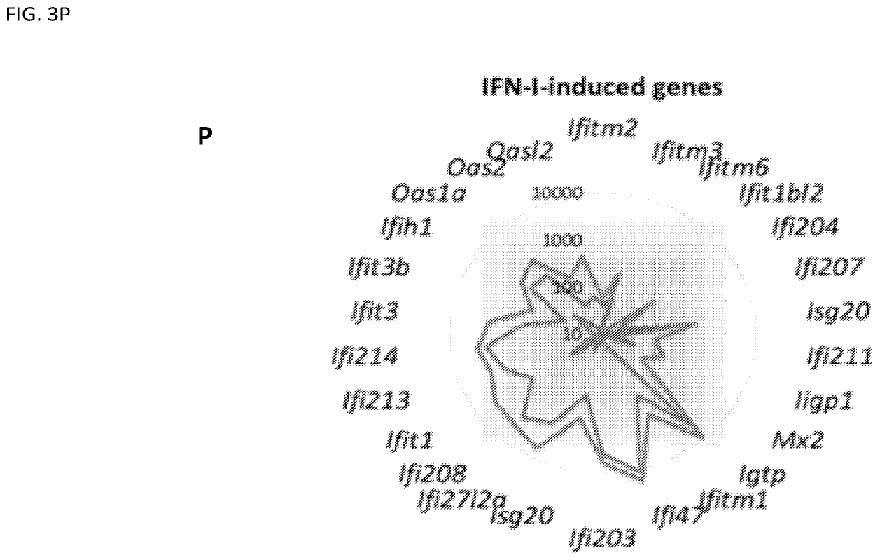

We analyzed gene expression on virus-specific CD8 T cells at the peak of the response, 7 d after acute infection with LCMV (FIG. 3, A and B). Short-term IFN-I blockade induced increased CD44 expression at the gene level (FIG. 3, C and D) and at the protein level (FIG. 3E) relative to control. IFN-I blockade also induced enriched expression of TCR signaling genes by Ingenuity Pathway Analyses (IPA; FIG. 3F) and costimulation genes by IPA and gene set enrichment analyses (GSEA; FIG. 3, F and G). Paradoxically, IFN-I-driven genes and IFN-I levels were increased in mice that received IFN-I blockade earlier during infection (FIG. 3, H-J), suggesting a “compensatory” IFNI response. There was also enrichment in IFNγ, IL-12, and IL-15 signaling genes in mice that received IFN-I blockade (FIG. 3, K-M), and these mice also exhibited increased activation and effector signatures (FIGS. 3, N and O). These data suggest that IFN-I blockade during the priming phase improved TCR and costimulation signaling later during the effector phase.

TCR triggering and costimulation are two main signals required for activation of the adaptive immune system. Furthermore, our data showing increases in IFN-I, IFNγ, IL-12, and IL-15 cytokine signatures also suggested an increase in the so-called “third signal” needed for the activation of T cell responses. We hypothesized that our short-term IFN-I blockade regimen could give a transient advantage to the virus, increasing antigen availability. To evaluate this, muscles were harvested at day 7 after intramuscular infection, and viral loads were quantified by plaque assays. Complete viral clearance was observed at day 7 in all mice, demonstrating that short-term IFN-I blockade does not prevent the resolution of acute viral infection, which normally occurs within a week (FIG. S3, A-C). Since IFN-I plays an early antiviral effect shortly after viral encounter, we hypothesized that IFN-I blockade could increase viral burden at very early time points following infection. To test this, we performed plaque assays at earlier time points 72 h after infection. Consistent with our hypothesis, IFN-I blockade resulted in a sharp increase in viral titers (FIG. S3, D-F). These data demonstrate that short-term IFN-I blockade induces a transient hyperacute burst in viral antigen, which is then rapidly cleared from the body.

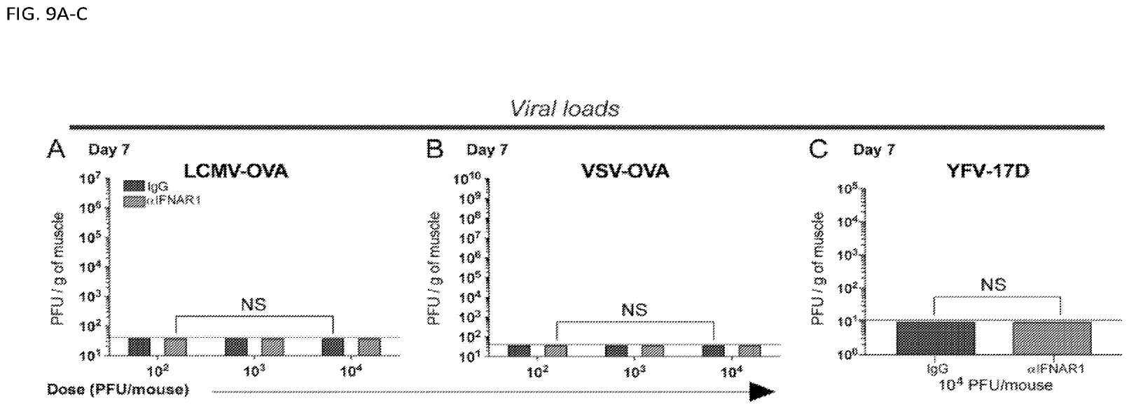

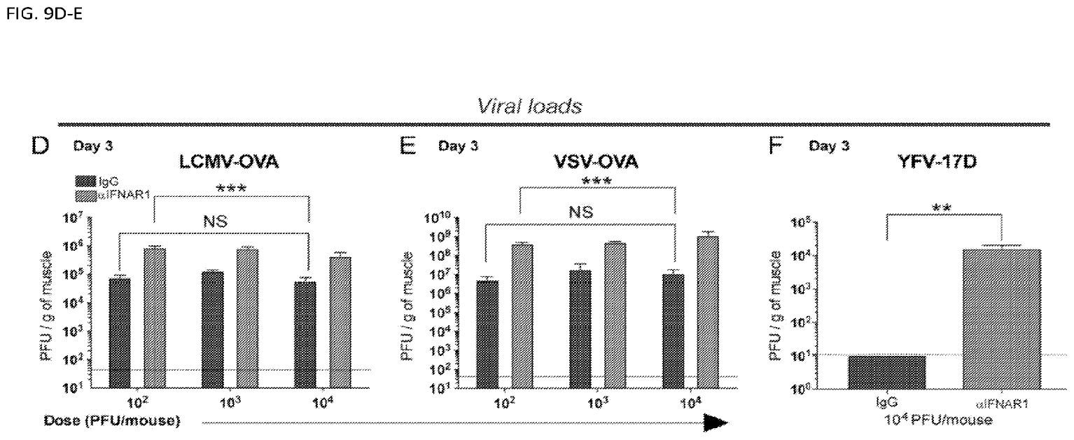

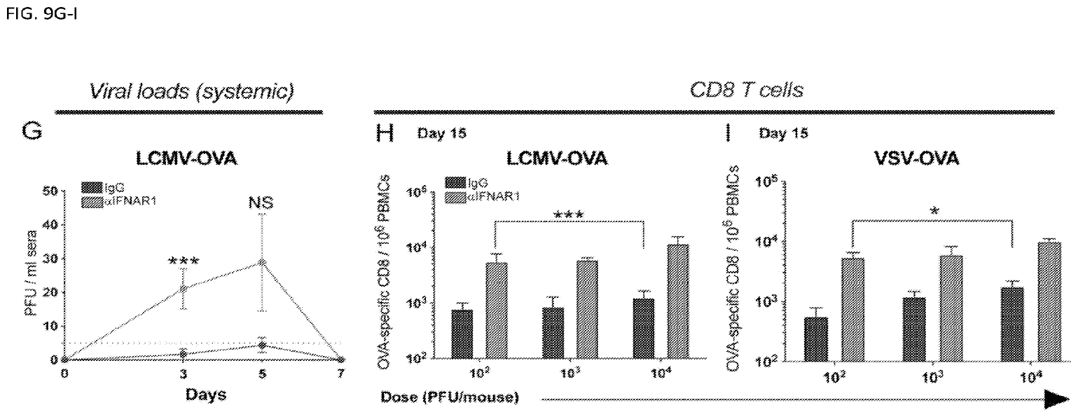

A prior study in individuals receiving the YFV-17D vaccine showed a positive correlation between vaccine replication and T cell responses (Akondy et al., 2015). Namely, individuals with the highest level of vaccine replication showed the most potent T cell response following vaccination. A logical prediction from that study is that increasing the dose of the vaccine would increase antigen levels and subsequent immunogenicity. However, we show that increasing vaccine dose in control mice (from 102 to 104 PFU, blue bars) does not significantly augment early viral loads 72 h after LCMV infection (FIG. 9D). Similar virologic effects were observed after vesicular stomatitis virus (VSV) vaccination and YFV-17D vaccination (FIG. 9, E and F). These results show that there is a strict limit on how much antigen can be expressed at the site of infection. These data also highlight that antigen levels depend mostly on the exponential replication of the virus, which critically depends on the translational machinery of infected cells. Since protein synthesis is inhibited by IFN-stimulated genes (Schoggins, 2019), this can help explain why injecting more virus did not substantially increase acute antigen loads. It is important to highlight that, following intramuscular infection, most of the virus replicates in situ, and very low levels of virus were detected in circulation. Systemic viral load was also resolved by day 7 in all mice (FIG. 9G). This demonstrates that short-term IFN-I blockade does not abrogate the ultimate clearance of the viral infection within a week. We also show that increasing viral dose does not significantly increase immunogenicity (FIG. 9, H and I). In summary, our findings demonstrate that short-term IFN-I blockade increases early antigen availability and immunogenicity in a way that cannot be recapitulated simply by increasing viral dose.

Mechanism: Effects of IFN-I Blockade on Antigen Presentation and Costimulation

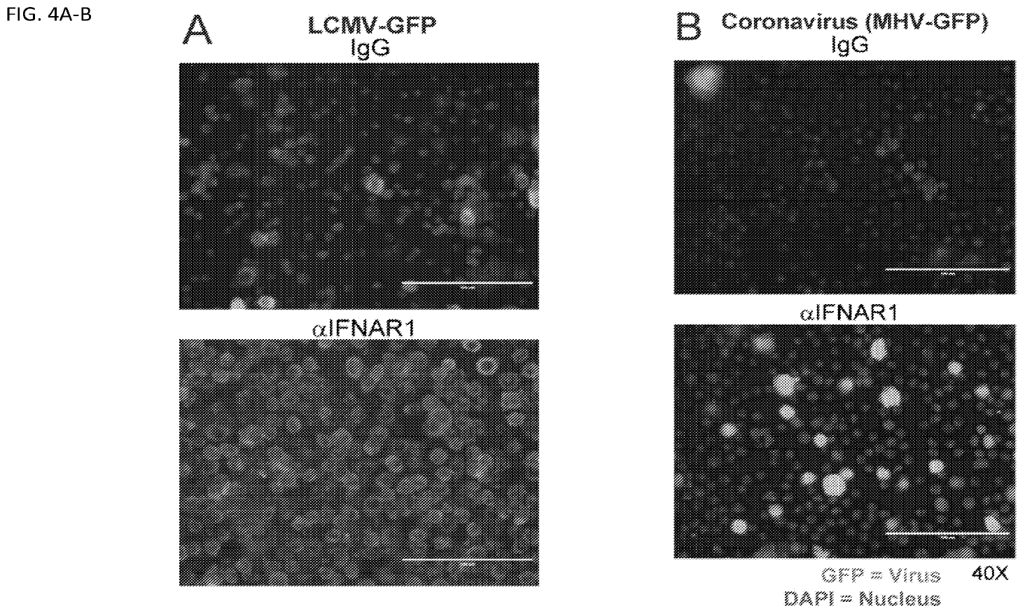

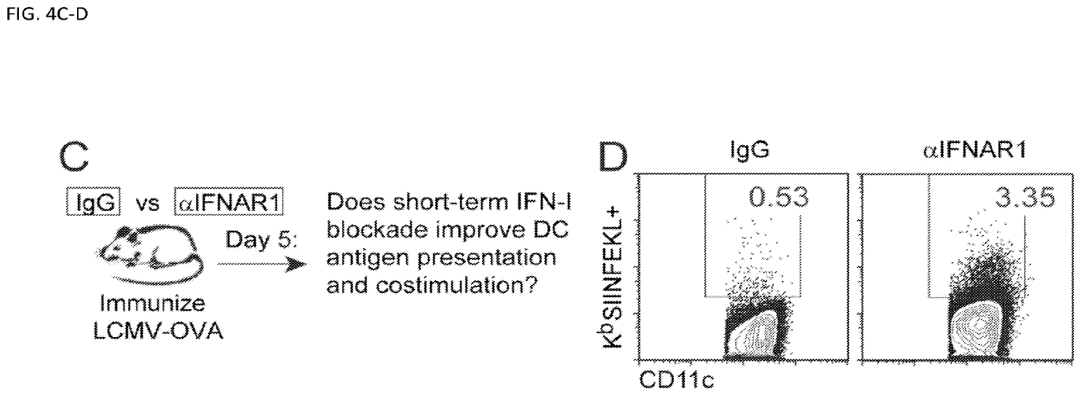

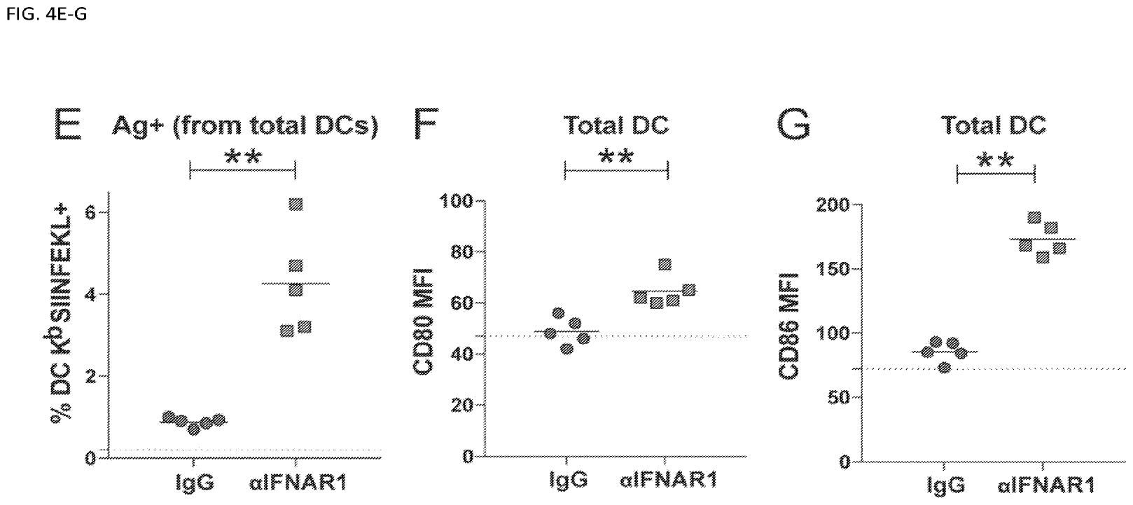

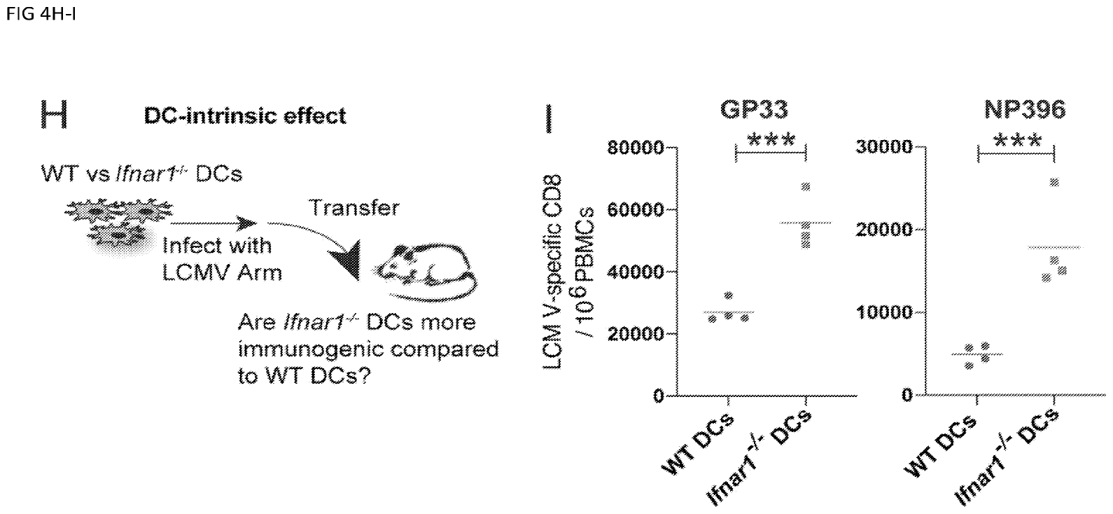

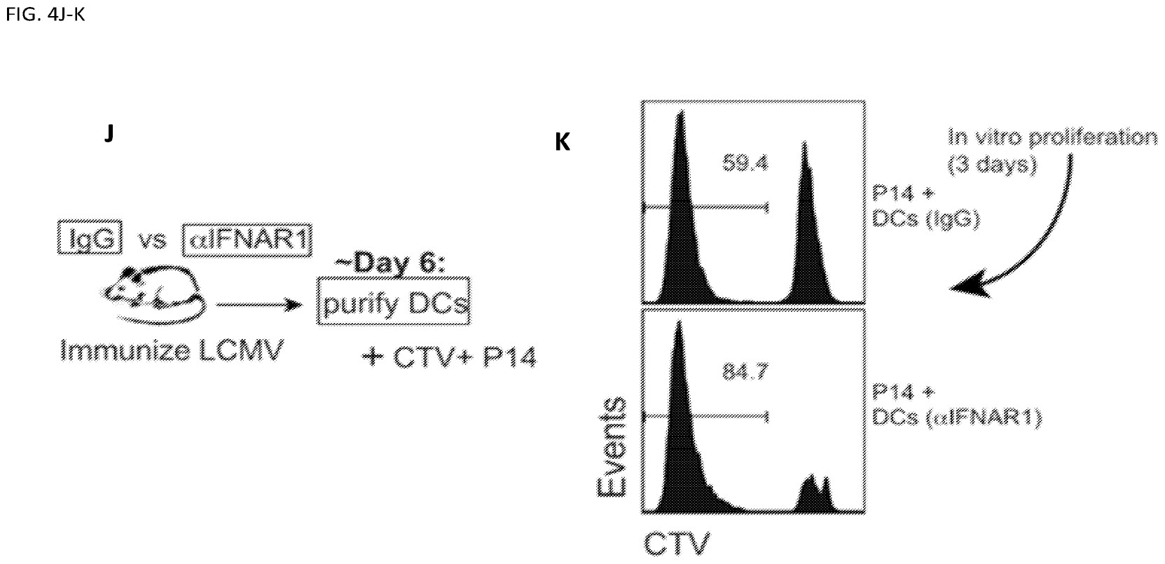

Our plaque assay data shown above demonstrate that short-term IFN-I blockade induces a transient increase in viral antigen 72 h after infection. Those experiments, however, did not specifically measure antigen loads in antigen-presenting cells, which are critical for the induction of adaptive immunity. We thus examined the effects of IFN-I blockade on dendritic cells (DCs) using an in vitro infection system. We cultured DCs with recombinant LCMV or MHV coronavirus-expressing GFP, with or without IFN-I blockade, and then we evaluated GFP expression on DCs. Our results show that IFN-I blockade resulted in more copious infection foci relative to control (FIG. 4, A and B). We also interrogated whether DCs from mice that received short-term IFN-I blockade were more effective at presenting cognate antigen and expressing costimulatory molecules (FIG. 4C). DCs from mice that received IFN-I blockade during primary viral infection showed higher levels of MHC-I molecules presenting cognate antigen (FIG. 4, D and E) and expressed higher levels of costimulatory molecules (FIG. 4, F and G) relative to control. At first glance, these data seemed counterintuitive, given that IFN-I signaling is a positive regulator of antigen presentation and costimulation. However, as shown earlier, blockade of the IFN-I pathway lasted only ˜96 h, and this was followed by a compensatory IFN-I response (FIG. 3, H-J), which could have explained the increase in antigen presentation and costimulation by DCs after day 5.

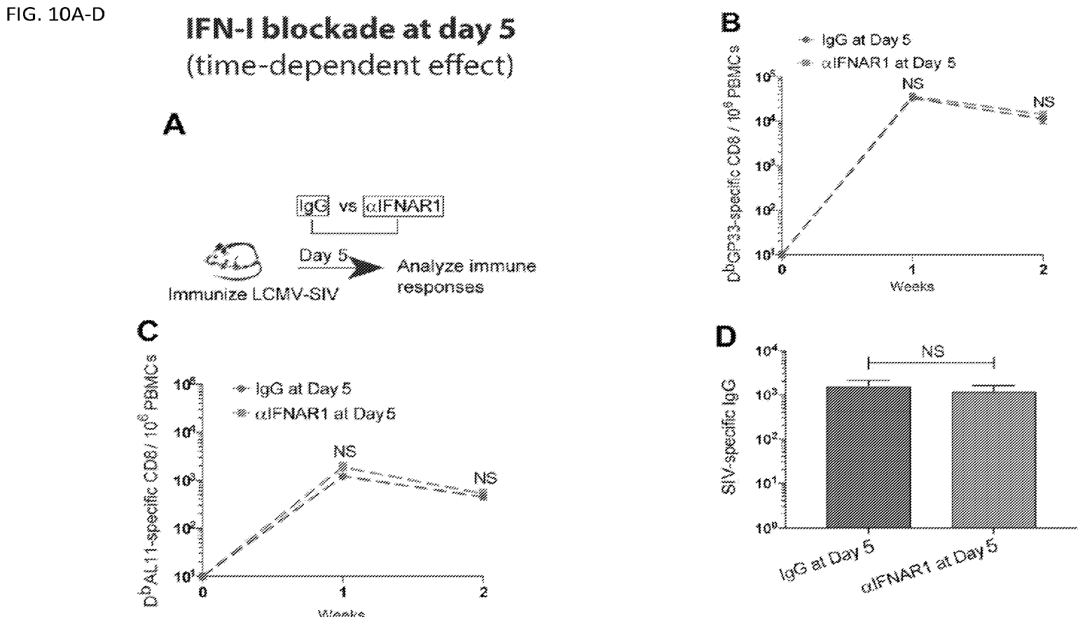

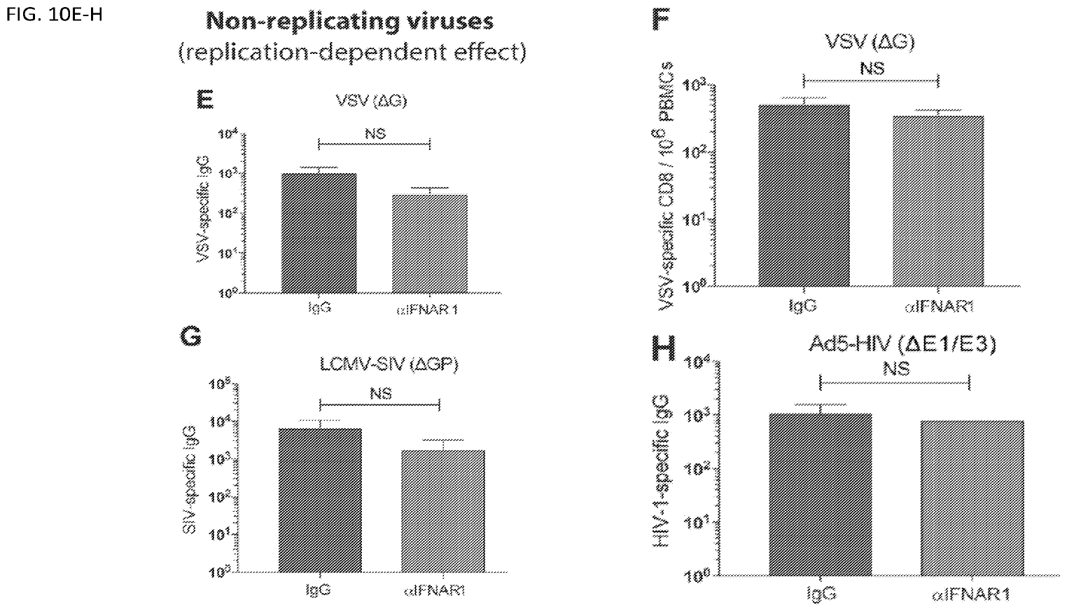

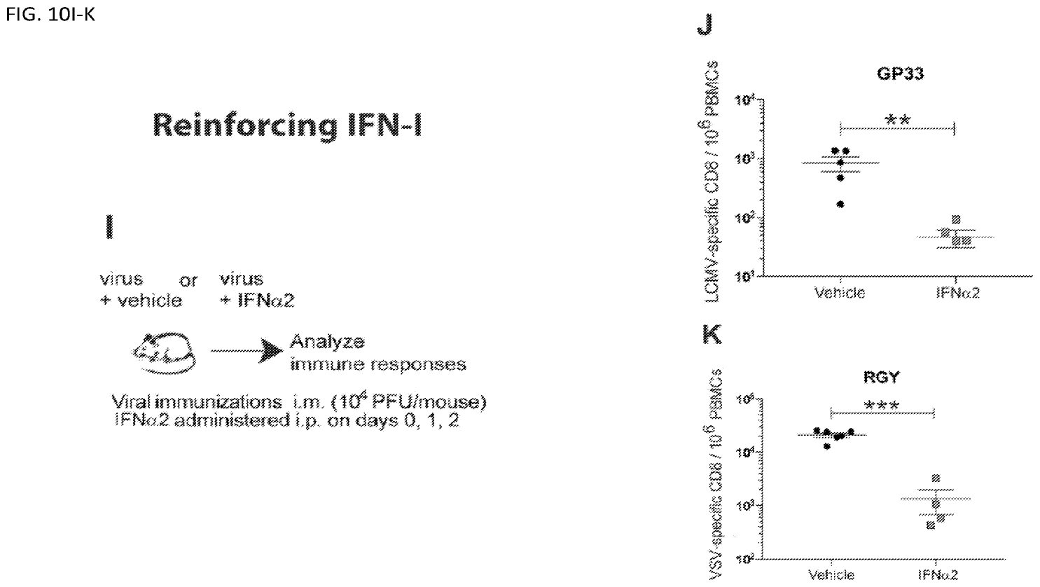

The IFN-I receptor is widely expressed on many cells, including DCs, which are also major producers of IFN-I (Reizis et al., 2011). This motivated us to study the DC-intrinsic effects of IFN-I signaling. We evaluated whether the absence of IFN-I signaling specifically on DCs would phenocopy the effect of short-term IFN-I blockade. We performed DC vaccinations using Ifnar1−/− DCs infected 24 h earlier with LCMV Armstrong (FIG. 4H). Strikingly, transfer of Ifnar1−/− DCs resulted in greater CD8 T cell responses relative to wild-type DCs (FIG. 4I), suggesting that IFN-I blockade modulated DC function. We also performed adoptive transfers of LCMV-specific T cells lacking IFNAR1 (Ifnar1−/−) followed by acute LCMV infection, and, consistent with prior reports (Kolumam et al., 2005; Havenar-Daughton et al., 2006), the permanent absence of IFN-I signaling on T cells resulted in long-term impairment of these responses (data not shown). According to prior studies, this could be explained by NK (Natural killer) cell-mediated killing of activated Ifnar1−/− T cells (Crouse et al., 2014; Xu et al., 2014). These earlier studies indicate that IFN-I signaling is intrinsically required for T cell immunity. Furthermore, we evaluated whether the adjuvant effect of IFN-I blockade was time dependent. We show that IFN-I blockade at day 5 after infection has no effect (FIG. 10, A-D), likely because IFN-I production normally subsides within 2-3 d of acute viral infection (FIG. 3J; Norris et al., 2013; Zuniga et al., 2008). IFN-I exert antiviral effects by regulating various biological processes; for example, by inhibiting protein translation in infected cells or by limiting a second round of infection in adjacent cells (Bailey et al., 2014; Farrell et al., 1978; McMichael et al., 2018).

To determine if the adjuvant effect of short-term IFN-I blockade was dependent on the latter process, we immunized mice with single-round (non-replicating) viruses. We first used a VSV vector that can enter cells and translate viral proteins but cannot induce a second round of infection due to genetic absence of the VSV G protein. Interestingly, short-term IFN-I blockade did not improve the immunogenicity of this virus (FIG. 10, E and F). Similar effects were reported with non-replicating LCMV and Ad5 viruses (FIG. 10, G and H). Therefore, the adjuvant effect of IFN-I blockade is mechanistically dependent on whether the virus can cause secondary foci of infection. Until now, our experiments have involved IFN-I blockade, and we next interrogated whether IFN-I supplementation would have an opposite effect (FIG. 10I). Systemic administration of IFN-I throughout the first 48 h of infection impaired primary immune responses (FIG. 10, J and K), consistent with a prior study that evaluated the effect of IFN-I supplementation (Honke et al., 2012). Collectively, these data suggest that the potent adjuvant effect of short-term IFN-I blockade is dependent on the timing and the ability of the virus to undergo additional rounds of infection.

As shown earlier, short-term IFN-I blockade increases the total number of memory CD8 T cells, and the next question was if this is caused by preferential expansion of specific memory subsets. To answer this question, we immunized mice with VSV with or without IFN-I blockade, and then we immune-phenotyped virus-specific CD8 T cells after 60 d. Consistent with our prior results, short-term IFN-I blockade increased the numbers of virus specific CD8 T cells in tissues (FIG. 5, A and B). Interestingly, most of the increase in the IFN-I blockade group was due to an increase in effector memory CD8 T cells (FIG. 5, C-G). A salient feature of effector memory CD8 T cells is their response-ready state (Wherry et al., 2003), which can provide rapid sterilizing protection following subsequent reinfections, especially in the context of highly replicating pathogens. This motivated us to perform challenge studies.

IFN-I Blockade During Viral Prime Improves Host Protection Following Future Reinfections

Does IFN-I blockade during an initial viral prime improve host protection after subsequent reinfections? To answer this simple question, we immunized mice with or without IFN-I blockade, and after several weeks we challenged mice with the same virus or a related pathogen to measure anamnestic immune protection. We used different challenge models to evaluate generalizability and to assess the contribution of different arms of the adaptive immune response in anamnestic immune protection.

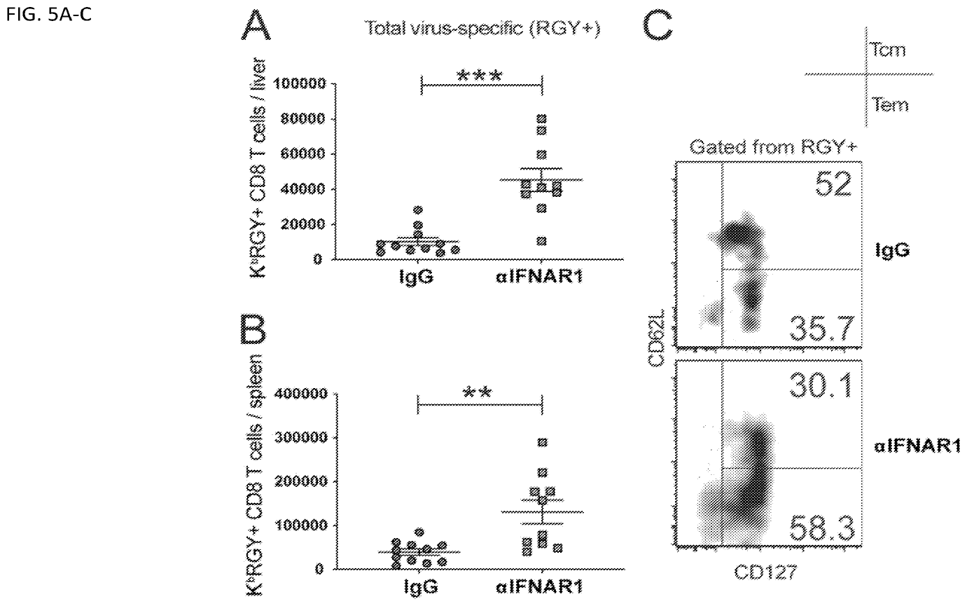

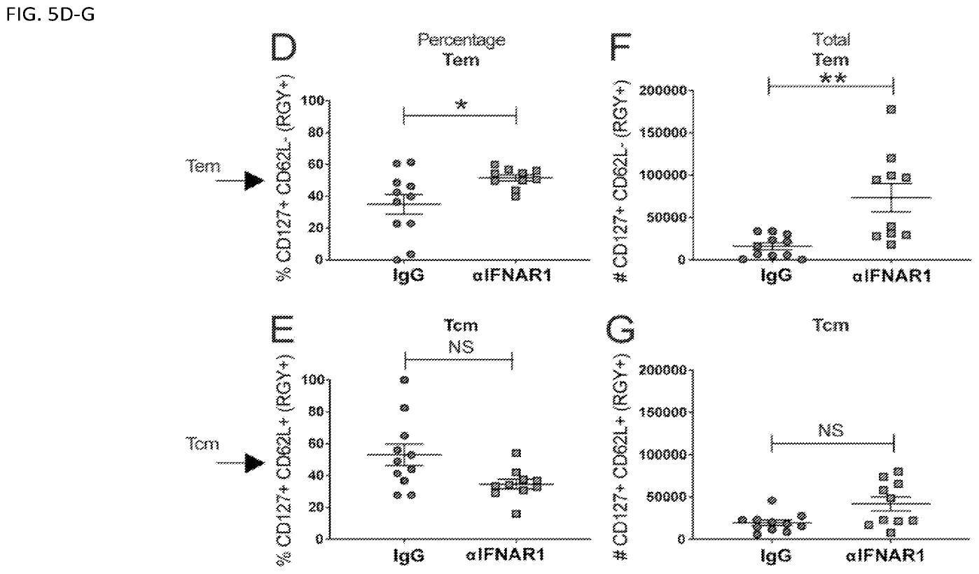

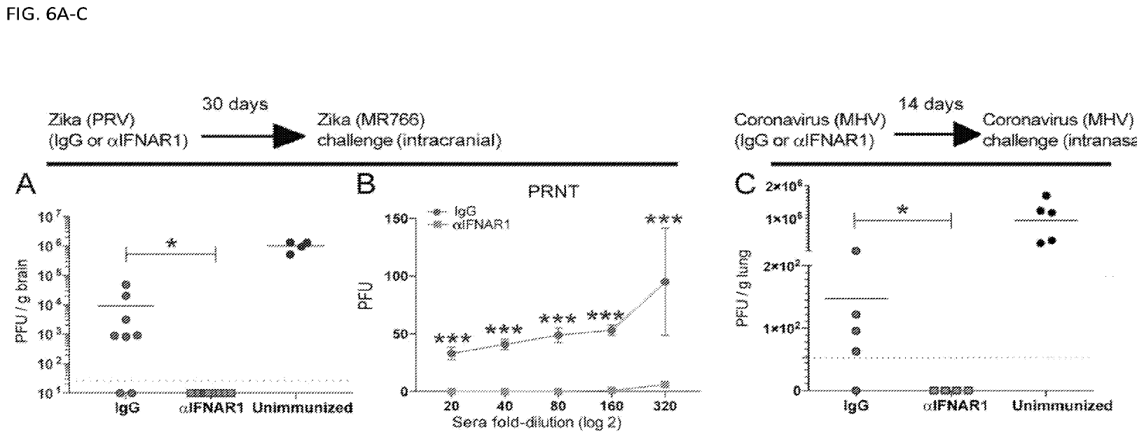

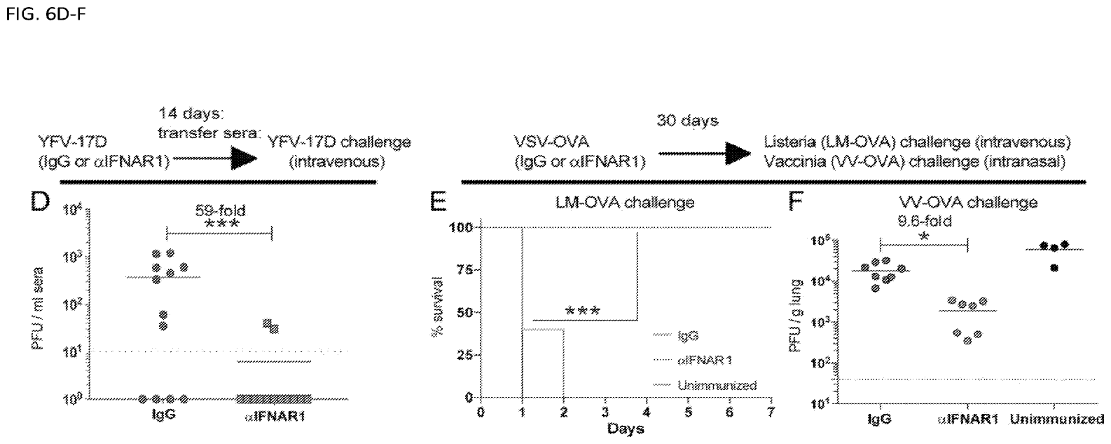

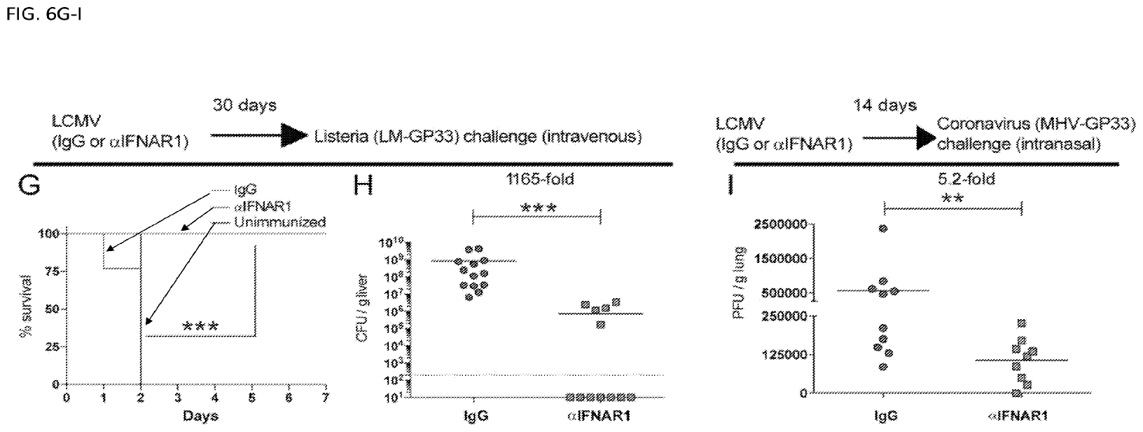

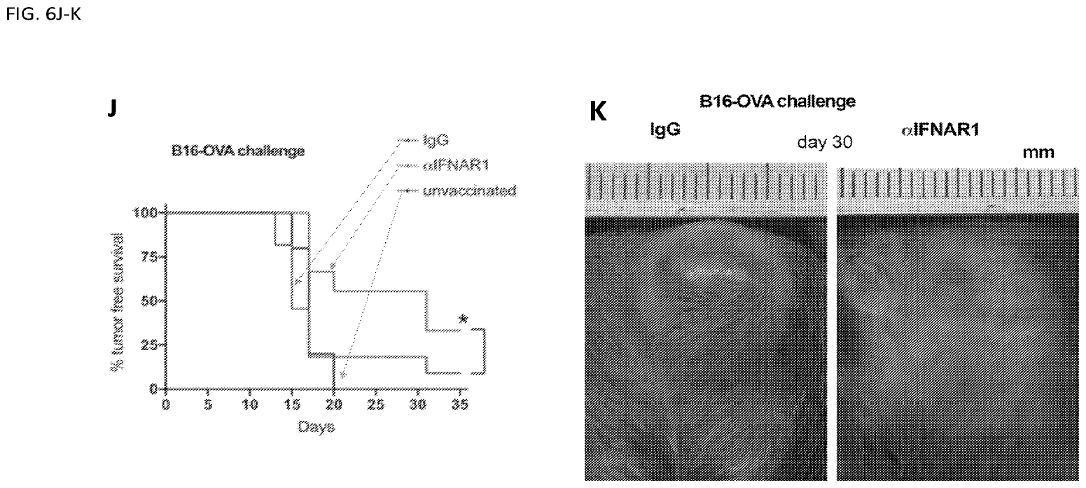

In our first challenge model, mice were immunized with Zika (Puerto Rico virus [PRV] strain) with or without IFN-I blockade. After 30 d, mice were challenged intracranially with different Zika (MR766 strain) to measure cross-protection. As expected, control Zika-immune mice showed only partial protection after heterologous Zika re-challenge when compared with unimmunized mice (FIG. 6A). But mice that received an initial Zika infection with IFN-I blockade exhibited sterilizing immunity after subsequent heterologous Zika challenge (FIG. 6A). These mice also exhibited improved antibody neutralization capacity by plaque reduction neutralization titer assays, with significant neutralization of heterologous Zika virus even at 320-fold sera dilution (FIG. 6B).

In our second challenge model, we interrogated whether a primary coronavirus infection with short-term IFN-I blockade would improve protection to coronavirus reinfection. In control immune mice, prior exposure to coronavirus conferred partial protection upon coronavirus re-challenge, but only 20% of mice exhibited sterilizing immunity (FIG. 6C). However, 100% of the mice that received IFN-I blockade during the primary coronavirus infection exhibited sterilizing immunity following subsequent coronavirus reinfections (FIG. 6C).

In our third challenge model, we used the clinically approved YFV-17D vaccine with or without IFN-I blockade. Since immunized wild-type mice are highly resistant to YFV-17D infection, we used a passive immunization model using Ifnar1−/− recipient mice, which are highly susceptible to YFV-17D (Erickson and Pfeiffer, 2013). We first immunized wild-type mice with YFV-17D intramuscularly with or without IFN-I blockade. After 2 wk, we transferred immune sera from these mice into naive Ifnar1−/− recipient mice, followed by systemic challenge with YFV-17D. Strikingly, the sera of mice that received IFN-I blockade during prior vaccination conferred sterilizing immunity in most recipient mice (FIG. 6D).

In our fourth challenge model, mice were immunized with a VSV-OVA vaccine and then challenged 30 d later with a supra Pfeiffer, 2013). We first immunized wild-type mice with YFV-17D intramuscularly with or without IFN-I blockade. After 2 wk, we transferred immune sera from these mice into naive Ifnar1−/− recipient mice, followed by systemic challenge with YFV-17D. Strikingly, the sera of mice that received IFN-I blockade during prior vaccination conferred sterilizing immunity in most recipient mice (FIG. 6D). In our fourth challenge model, mice were immunized with a VSV-OVA vaccine and then challenged 30 d later with a supralethal dose of Listeria monocytogenes (LM)-expressing OVA (LMOVA). Control immune mice succumbed to this bacterial challenge, whereas mice that had previously received IFN-I blockade during the initial VSV-OVA prime survived the subsequent LMOVA challenge (FIG. 6E). Enhancement of immune protection was also observed after a viral challenge with vaccinia virus (VV)-expressing OVA (FIG. 6F).

In our fifth challenge model, mice were immunized with LCMV, and after 30 d, mice were challenged with a supra-lethal dose of LM expressing a CD8 T cell epitope derived from LCMV (LM-GP33). As expected, all naive and control immune mice succumbed rapidly to this supra-lethal bacterial challenge. However, all of the mice that received IFN-I blockade during the initial LCMV prime 30 d earlier survived the subsequent LM-GP33 challenge (FIG. 6G), with 58% of animals exhibiting sterilizing immunity (FIG. 6H). Finally, we used a model for coronavirus vaccination in which mice are first vaccinated with an LCMV vector, and after several weeks, they were challenged intranasally with coronavirus expressing a CD8 T cell epitope from LCMV (MHV-GP33). Interestingly, IFN-I blockade during an initial vaccination conferred a fivefold improvement in immune protection against coronavirus (FIG. 6I).

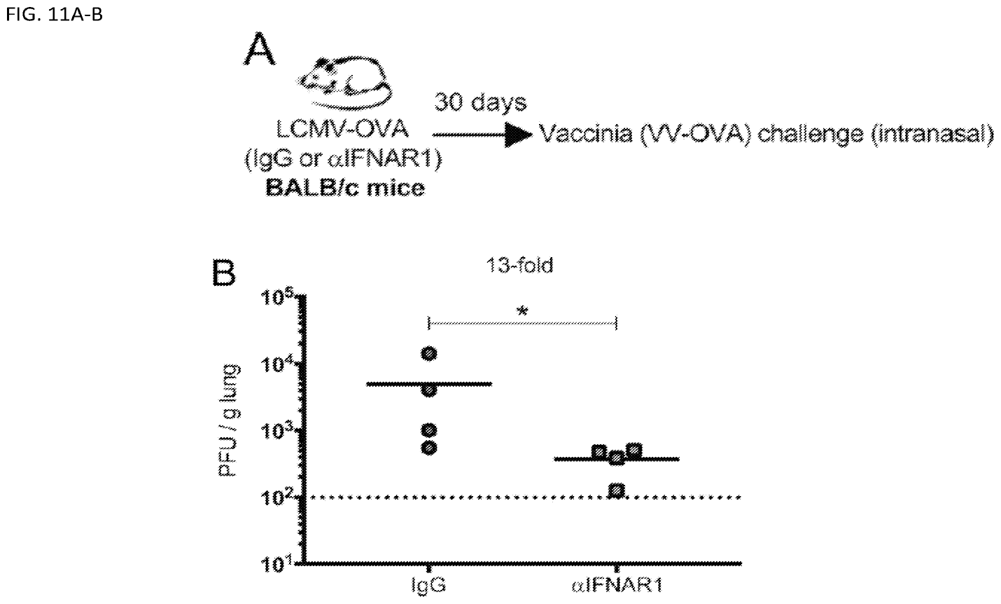

Collectively, these data show that short-term IFN-I blockade during an initial viral infection or viral vaccination renders the host better protected against subsequent infections with similar or related pathogens. Note that most challenge models used in FIG. 6 evaluated overall protection conferred by both cellular and humoral responses. However, the experiments in FIG. 6D specifically evaluated protection by humoral responses, whereas the experiments in FIG. 6, G-I, specifically evaluated protection by CD8 T cell responses (since only a CD8 epitope was matched between the primary and secondary infection). These results were all using C57BL/6 mice, but similarly, BALB/c mice also showed improved anamnestic protection when short-term IFN-I blockade was administered during the initial viral prime (FIG. 11). Altogether, our studies using different viruses, routes, challenges, and host genetic backgrounds show that a transient blockade of IFN-I signaling during an initial virus encounter can improve immunological memory and protection against future reinfections. Such results demonstrate a novel finding: short term IFN-I blockade during an initial viral prime induces a long-term improvement in immunological memory. This positive effect was reproduced among different acute viral infections as well as vaccinations.

Discussion

It is widely accepted that IFN-I play a critical role in the generation of immunological memory following acute infection or vaccination. However, most studies on IFN-I have focused on the “all-or-none” effects of IFN-I, for example, in the context of IFNAR1 genetic mutations that permanently impair IFN-I sensing or in the setting of long-term IFN-I blockade (Hernandez et al., 2019; Kolumam et al., 2005; Le Bon et al., 2003; Müller et al. 1994; Sandler et al., 2014; Teijaro et al., 2013; Wilson et al., 2013). Therefore, the effects of blocking IFN-I short term, specifically at the time of viral prime, are not well studied.

Here, we demonstrate an unexpected favorable effect of blocking IFN-I short term. Prior studies have also evaluated the effects of IFN-I blockade in the context of immune exhaustion caused by chronic infections. Elegant papers by Brooks, Oldstone, and others have shown that IFN-I blockade can ameliorate immune exhaustion during chronic LCMV infection, suggesting that IFN-I play a negative role during chronic LCMV infection. This effect is not observed in other chronic viral infections (HIV, SIV, and Hepatitis C virus) in which IFN-I are thought to play a positive role, suggesting virus-dependent effects (Torriani et al., 2004; Azzoni et al., 2013; Teijaro et al., 2013; Wilson et al., 2013; Sandler et al., 2014; Bhattacharyya et al., 2017; Cheng et al., 2017). Our study is conceptually different than prior studies because we elucidate the effects of IFN-I blockade in the context of immunological memory, focusing on how short-term modulation of this pathway, specifically at the time of prime, affects susceptibility to future reinfections. We demonstrate across multiple viral systems that short-term blockade of IFN-I improves immunological memory and anamnestic immune protection. Notably, this positive effect was also extended to clinically approved vaccines, including YFV-17D, as well as experimental HIV-1 vaccines. In this setting, we show that short-term IFN-I blockade improved the immune coverage afforded by HIV vaccines, rendering antibody responses better able to recognize variant envelopes from different HIV clades.

Although IFN-I-modulated vaccines seemed safe in mice, future studies are needed to evaluate safety in primates. IFN-I blockers have already been used in humans to ameliorate inflammatory diseases and have shown acceptable safety profiles (Casey et al., 2018; Felten et al., 2019; Tanaka et al., 2020). A reasonable counterargument against IFN-I-modulated vaccines, however, is that increasing vaccine replication creates a safety concern. Nevertheless, all acute viruses and vaccines were completely cleared within a week of injection. Moreover, short term IFN-I blockade was followed by a compensatory IFN-I response, likely caused by a feedback mechanism and the transient overload in antigen. Therefore, a single dose of αIFNAR1 administered during the viral prime did not really “block” IFN-I responses per se; it just delayed the IFN-I response and actually reinforced it later throughout the effector phase of the immune response. Interestingly, a prior study showed that “pre-emptive IFN-I stimulation” before T cell priming, known as out of sequence signal, impairs T cell activation (Welsh et al., 2012). This suggests an additional mechanism by which delaying IFN-I signaling may improve immune responses. Furthermore, we reason that the late IFN-I response likely contributes to the improved memory response because if the IFN-I pathway is absent long term, adaptive immune responses are severely impaired and the host is unable to clear the acute viral infection (Teijaro et al., 2013; Wilson et al., 2013). Antigen is necessary for the elicitation of adaptive immunity. However, high antigen loads for a prolonged time induces inhibitory mechanisms and is thus a main reason for immune exhaustion during chronic infection (Gallimore et al., 1998; Mueller and Ahmed, 2009; Penaloza-MacMaster et al., 2014; Penaloza-MacMaster, 2017).

It is important to highlight that short-term IFN-I blockade instituted a different immune scenario: a drastic increase in antigen followed by rapid antigen control. These data suggest that a transient overload in antigen levels “raises the alarm” on the immune system, improving antigen presentation and costimulation at a critical time when the adaptive immune system is getting primed. In particular, a transient increase in antigen levels during the first 72 h of a viral infection can induce a long-term potentiation of immunological memory. These findings have not yet been validated in humans, but previous clinical studies demonstrate a positive correlation between vaccine replication and vaccine immunogenicity (Akondy et al., 2015; Lin et al., 2020). Therefore, a logical assumption is that increasing vaccine dose would proportionally increase immunogenicity, but this is not the case. We show that increasing vaccine dose above a certain level does not significantly augment immunogenicity because there is a natural limit on how much antigen can be expressed by infected cells, which is critically influenced by IFN-I and not so much by the initial virus inoculum. Enhancing adaptive immunity by blocking an innate immune pathway seemingly violates the classical paradigm that a potent innate response gives rise to a potent adaptive response (Braciale and Hahn, 2013; Kadowaki et al., 2000; Medzhitov and Janeway, 1998; Pulendran et al., 2013). However, these two arms of the immune system do not always work in cooperation. The innate protection conferred by IFN-I has been honed over millions of years to control the initial dissemination of viruses, conferring an immediate survival benefit to the host. Nevertheless, acute IFN-I responses can extinguish viral antigen prematurely, curtailing the elicitation of immunological memory and thus limiting the future protection of the host. In conclusion, we show that co-administration of viral vaccines together with an IFN-I blocker results in profound improvement of immunological memory. Although the safety of IFN-I-modulated vaccines would need careful validation in primates, these findings provide insights for rational vaccine design, as well as a framework to understand the tug of war between innate immunity and immunological memory.

Materials and Methods

Mice, Treatments, Infection, and Challenges

For Zika infection experiments, we used 4-wk-old mice (C57BL/6) since this facilitated intracranial challenges. In all other experiments, 6-8-wk-old C57BL/6 or BALB/c mice were used. Mice were purchased from The Jackson Laboratory (approximately half males and half females). Mice were immunized intramuscularly (50 μl per quadriceps), subcutaneously (100 μl in the right flank), or intranasally (25 μl per nostril) with the indicated viral vectors. IgG isotype control (MOPC-21) or IFN-I receptor subunit 1 (IFNAR1)-blocking antibodies (MAR1-5A3) were purchased from BioXCell or Leinco and diluted in sterile PBS. 100 μg of antibody was administered, admixed together with each viral vector vaccine (as a single bolus). Zika challenges were performed intracranially with 10⁴ PFU. Supra-lethal bacterial challenges were performed with either LM-GP33 or LM-OVA at 10⁷ CFU intravenously via lateral tail vein injection using a mouse restrainer. Other viral challenges consisted of 2×10⁶ PFU of VV-OVA through the intranasal route, 10⁶ PFU of YFV-17D through the intravenous route, or 10⁶ PFU of MHV coronavirus through the intranasal route. Mice were housed at the Northwestern University Center for Comparative Medicine located in downtown Chicago. All mouse experiments were performed with approval of the Northwestern University Institutional Animal Care and Use Committee.

Adoptive Cell Transfers and DC Analyses

For in vivo DC transfers, we generated bone marrow-derived DCs using a protocol similar to prior publications (Penaloza-MacMaster et al., 2015; Wang et al., 2019). In brief, bone marrow cells from wild-type or Ifnar1−/− mice were cultured for 5 d in GM-CSF (Sigma) at 20 ng/ml to generate DCs. On day 5, the media were aspirated and DCs were infected with LCMV Armstrong at a multiplicity of infection of 0.05 in 1% FBS RPMI, gently rocking every 10 min. After 1 h, media were replaced with 10% FBS DMEM. After 1 d of in vitro infection, DCs were washed five times and injected intravenously into naive mice (2.5×105 DCs/mouse). CD8 T cell responses were evaluated after specific DC transfer. In FIG. 4, A and B, we used a DC cell line (DC 2.4) for imaging.

Reagents, Flow Cytometry, and Equipment

Single-cell suspensions were obtained from peripheral blood mononuclear cells (PBMCs) and tissues as previously described (Masopust et al., 2001). Dead cells were gated out using LIVE/DEAD fixable dead cell stain (Invitrogen). The HIV peptide pools used for intracellular cytokine staining were obtained from the AIDS Reagent Resource, and all other peptides were from AnaSpec or GenScript. MHC class I tetramers were obtained from the National Institutes of Health (NIH) tetramer facility at Emory University. Cells were stained with anti-CD8a (53-6.7 on PerCP-Cy5.5), anti-CD44 (IM7 on Pacific Blue), H-2Kb SIINFEKL (eBio25-D1.16 on APC), CD80 (16-10A1 on FITC), TNFα (MP6-XT22 on PE-Cy7), IL-2 (JES6-5H4 on PE), IFNγ (XMG1.2 on APC), peanut agglutinin (conjugated to fluorescein), Fas (Jo2 on PE), IgD (11-26 on Pacific Blue), IgM (RMM-1 on PE-Cy7), B220 (RA3-6B2 on PerCP-Cy5.5), IFNAR1 (MAR1-5A3 on PE), and CD3 (145-2c11 on FITC). Fluorescently labeled antibodies were purchased from BD PharMingen, except for CD44 (which was from Biolegend). Flow cytometry samples were acquired with a Becton Dickinson Canto II or an LSRII and analyzed using FlowJo (Treestar).

Virus-Specific ELISA

Virus-specific ELISA was done as described in prior publications (Dangi et al., 2020; Wang et al., 2019). In brief, 96-well flat bottom plates (MaxiSorp; Thermo Scientific) were coated with 100 μl/well of the respective viral lysate (e.g., Zika, YFV-17D, VSV, and MHV) diluted 1:10 in PBS for 48 h at room temperature. Plates were washed with PBS+0.5% Tween 20. Blocking was performed for 2 h at room temperature with 200 μl of PBS+0.2% Tween 20+10% FCS. 5 μl of sera were added to 145 μl of blocking solution in the first column of the plate, 1:3 serial dilutions were performed until row 12 for each sample, and plates were incubated for 90 min at room temperature. Plates were washed three times followed by addition of goat anti-mouse IgG conjugated to horseradish peroxidase (Southern Biotech) diluted in blocking solution (1:5,000) at 100 μl/well and incubated for 90 min at room temperature. Plates were washed three times, and 100 μl/well of Sure Blue substrate (SeraCare) was added for 8 min. Reaction was stopped using 100 μl/well of KPL TMB Stop Solution (SeraCare). Absorbance was measured at 450 nm using a Spectramax Plus 384 (Molecular Devices).

Protein-Specific ELISA

96-well flat-bottom plates (MaxiSorp; Thermo Scientific) were coated with 0.1 μg/well of gp140 derived from SIVmac239 (ImmuneTech), HIV-1 clade A BGB505 (ImmuneTech), or HIV-1 clade B SF162 (ImmuneTech); or OVA (Worthington) for 48 h at 4° C. Plates were washed with PBS+0.05% Tween 20. Blocking was performed for 4 h at room temperature with 200 μl of PBS+0.05% Tween 20+bovine serum albumin. 6 μl of sera were added to 144 μl of blocking solution in the first column of the plate, 1:3 serial dilutions were performed until row 12 for each sample, and plates were incubated for 60 min at room temperature. Plates were washed three times followed by addition of goat anti-mouse IgG conjugated to horseradish peroxidase (Southern Biotech) diluted in blocking solution (1:5,000) at 100 μl/well and incubated for 60 min at room temperature. Plates were washed three times, and 100 μl/well of Sure Blue substrate (SeraCare) was added for 8 min. Reaction was stopped using 100 μl/well of KPL TMB Stop Solution (SeraCare). Absorbance was measured at 450 nm using a Spectramax Plus 384 (Molecular Devices).

TZM-b1 Assays

TZM-b1 cells (a HeLa cell line engineered to express human receptors and coreceptors for HIV, in addition to a Tat-inducible luciferase gene) were used to measure vaccine-induced antibody neutralization of SIV. TZM-b1 cells were cultured in T75 flasks (Thermo Scientific) at 10⁴ cell/ml density for 3 d in 10% FBS complete DMEM (GIBCO). On the day of the assay, 1:20 serial fold dilutions of mouse sera were performed. Sera were incubated with SIVmac251.TCLA pseudovirus for 30 min in low evaporation 96-well clear plates (Corning). TZM-b1 cells were detached from flasks using 0.25% trypsin-EDTA (GIBCO) and seeded at a density of 0.5×10⁶/ml per well. On the following day, 10% FBS complete DMEM (GIBCO) was added to each well. At day 3, media were aspirated, and cells were lysed using luciferase cell culture lysis buffer (Promega). Luciferase reaction was performed using 30 μl of cell lysis (Promega). The reaction was added to 96-well black optiplates (Perkin Elmer). Luminescence was measured using a Perkin Elmer Victor3 luminometer.

Bacterial Quantification

To quantify LM in liver, brain heart infusion agar containing 50 μg/ml streptomycin was prepared and added to 6-well plates (2 ml of agar/well). LM-GP33 and LM-OVA possess a streptomycin resistance gene. Liver was harvested and collected in 14-ml round-bottom tubes (Falcon) with 5 ml of 1% FBS DMEM (without antibiotics). Tissue was passed through a 100-μm strainer (Scientific Inc.) using 1% Triton X-100 solution. 50 μl of serial dilutions were added on each brain heart infusion agar well. Plates were incubated at 37° C. overnight. CFU were counted the next day.

Viral Quantification

Quantification of LCMV on Vero E6 cell monolayers was done as described in prior publications (Dangi et al., 2020; Wang et al., 2019). In brief, Vero E6 cells (ATCC) were grown on 6-well plates at 2×105 cells/ml. After cells reached ˜90% confluency, media were removed and 200 μl of 10-fold viral dilutions in 1% DMEM (GIBCO) were pipetted on top of the cell monolayers. Plates were rocked every 10 min in a 37° C., 5% CO₂ incubator. After 60 min, media were aspirated, and the monolayers were overlaid with a 1:1 solution of 2×199 media and 1% agarose. After 4 d of culture in a 37° C., 5% CO₂ incubator, a second overlay was added consisting of a 1:1 solution of 2×199 media and 1% agarose with neutral red. The agar overlay was removed on day 5, and plaques were counted using a transilluminator (Gradco). Quantification of VSV titers was similar to quantification of LCMV titers except that the agar overlay was removed after 1 d and 1% crystal violet was added on top of the monolayers, incubated for 1 h at room temperature, and then washed with H₂O. MHV viral quantification was similar to VSV, but overlay was removed 2 d after infection. Quantification of YFV-17D titers was similar to quantification of LCMV titers except that the agar overlay was removed after 5 d and 1% crystal violet was added on top of the monolayers, incubated for 1 h at room temperature, and then washed with H₂O. For quantification of vaccinia, we followed the protocol by Dr. Bernard Moss (Cotter et al., 2017). For viral load quantification in muscle, both quadriceps were harvested and collected in round-bottom tubes (Falcon) containing 3 ml of 1% FBS DMEM (GIBCO). Muscle tissue was processed using a Tissue Ruptor homogenizer (Qiagen). Lungs and brains were harvested and collected in round-bottom tubes (Falcon) containing 1 ml of 1% FBS DMEM (GIBCO) and homogenized as described above. Following homogenization, tissues were clarified using a 100-μm strainer (Scientific Inc.) to remove debris.

Viruses

LCMV-expressing SIVmac239 antigens (Gag and Env), GFP, or OVA were from Hookipa Biotech and were produced as described previously (Kallert et al., 2017). Non-replicating LCMV vectors were also from Hookipa Biotech. The non-replicating LCMV vectors contained the LCMV glycoprotein gene in trans, which resulted in a single round of infection (Penaloza-MacMaster et al., 2017). Replicating LCMV-HIV vectors were constructed with help from the De La Torre (Scripps Research Institute, San Diego, Calif.) and Waggoner (University of Cincinnati College of Medicine, Cincinnati, Ohio) laboratories. The non-replicating Ad5 vector is E1/E3 deleted and expresses HIV-1 gp140 V1-V3 domains from HIV-1 Bal, and was obtained from the Mascola laboratory (NIH, Bethesda, Md.). The VSVOVA used in this study was obtained from Dr. Vaiva Vezys (University of Minnesota Medical School, Minneapolis, Minn.) and was derived from a stock from Dr. Leo Lefrancois' laboratory (University of Connecticut Health Center, Farmington, Conn.). The nonreplicating VSV vector (VSV-ΔG) that contains the G protein in trans was a gift from Dr. Connie Cepko (Harvard Medical School, Boston, Mass.). The acute LCMV Armstrong strain was propagated from a stock from Dr. Rafi Ahmed's laboratory (Emory University, Atlanta, Ga.). Dr. Bernard Moss and Dr. Patricia Earl (NIH) provided the poxviruses. The following reagents were obtained through the NIH Biodefense and Emerging Infections Research Resources Repository, NIAID, and NIH (BEI Resources): YFV-17D, NR-115; Zika virus, PRVABC59 (Puerto Rico strain), NR-50240; Zika virus, MR766 (Uganda strain), NR-50065. Mouse betacoronaviruses (MHV-A59 strain) were a gift from Dr. Susan Weiss (University of Pennsylvania, Philadelphia, Pa.). Murine IFNα2 293F mammalian cells were transfected with a pcDNA3.1 mIFNα2 expression vector (Addgene ID 135098). IFNα2 purification was performed by cation exchange chromatography using a HiTrap SP column (General Electric). Purified recombinant mIFNα2 was quantified by an IFNα mouse ELISA kit (Invitrogen).

Immunofluorescence Staining

For immunofluorescence staining of DCs, the murine DC2.4 cell line was used. Cells were plated in clear flat-bottom 96-well plates at a density of 104 cells/well in 200 μl of 10% FBS DMEM (GIBCO), 1% L-glutamine, and 1% penicillin-streptomycin. After 2 d, cells were treated with 20 μg of IgG control or αIFNAR1 antibodies for 30 min. Media were removed from each well using a multichannel pipette, and cells were infected with LCMV-GFP or MI-IV-GFP (MOI 0.05) in 50 μl of 1% FBS DMEM (GIBCO) for 1 h, gently rocking every 10 min. Media were removed and replaced with 200 μl of 10% FBS DMEM (GIBCO), 1% L-glutamine, and 1% penicillin-streptomycin and incubated at 37° C. and 5% CO₂ for 72 h. Cells were washed once and fixed with 4% paraformaldehyde (Thermo Scientific). A Vectashield mounting medium containing DAPI (Vector Labs) was added to the wells, and images were acquired using an EVOS FL digital inverted microscope (Thermo Scientific).

RNA-Seq Data Acquisition and Analysis

Gene expression profiling was performed as shown previously (Barnitz et al., 2013; Penaloza-MacMaster et al., 2015; Quigley et al., 2010; Wang et al., 2019). In brief, C57BL/6 mice were intramuscularly immunized with 104 PFU of LCMV, and at day 7, splenic CD8 T cells were MACS (magnetic activated cell sorting) sorted with aMACS negative selection kit (STEMCELL). Purified CD8 T cells were stained with DbGP33 tetramer, live dead stain, and flow cytometry antibodies for CD8 and CD44 to gate on activated CD8 T cells. Live, CD8+, CD44+, and DbGP33+ cells were FACS sorted to ˜97% purity on a FACS Aria cytometer (BD Biosciences) and stored at −80° C. in 1 ml of TRIzol (Life Sciences). RNA extraction was performed with the RNAdvance Tissue Isolation kit (Agencourt). RNA quality and RNA-Seq downstream analyses were performed at the NUSeq core at Northwestern University. For analysis, adapters were trimmed from reads using cutadapt version 1.13 and aligned to 10 mm using STAR version 020201. For gene counting, htseq-count version 0.6.1p1 was used, and differential expression analysis was conducted using DESeq2 version 1.14.1. RNA-Seq data were uploaded into the GEO database (accession no. GSE129827) in a record titled “Gene expression comparison of splenic virus specific CD8 T cells after infection with LCMV vector and treatment with IgG or αIFNAR1.

Statistical Analysis

Most statistical analyses used the Mann-Whitney test, unless specified otherwise in the figure legend. Survival plot analyses were performed using the Mantel-Cox test. Dashed lines in plaque assay and ELISA plots represent the limit of detection. Data were analyzed using Prism (Graphpad).

### REFERENCES

Akondy, R. S., P. L. Johnson, H. I. Nakaya, S. Edupuganti, M. J. Mulligan, B. Lawson, J. D. Miller, B. Pulendran, R. Antia, and R. Ahmed. 2015. Initial viral load determines the magnitude of the human CD8 T cell response to yellow fever vaccination. Proc. Natl. Acad. Sci. USA. 112:3050-3055. https://doi.org/10.1073/pnas.1500475112.
Azzoni, L., A. S. Foulkes, E. Papasavvas, A. M. Mexas, K. M. Lynn, K. Mounzer, P. Tebas, J. M. Jacobson, I. Frank, M. P. Busch, et al. 2013. Pegylated Interferon alfa-2a monotherapy results in suppression of HIV type 1 replication and decreased cell-associated HIV DNA integration. J. Infect. Dis. 207:213-222. https://doi.org/10.1093/infdis/jis663.
Bailey, C. C., G. Zhong, I. C. Huang, and M. Farzan. 2014. IFITM-Family Proteins: The Cell's First Line of Antiviral Defense. Annu. Rev. Virol. 1: 261-283. https://doi.org/10.1146/annurev-virology-031413-085537.
Barnitz, R. A., S. Imam, K. Yates, and W. N. Haining. 2013. Isolation of RNA and the synthesis and amplification of cDNA from antigen-specific T cells for genome-wide expression analysis. Methods Mol. Biol. 979: 161-173. https://doi.org/10.1007/978-1-62703-290-2_13.
Bhattacharyya, M., P. Madden, N. Henning, S. Gregory, M. Aid, A. J. Martinot, D. H. Barouch, and P. Penaloza-MacMaster. 2017. Regulation of CD4 T cells and their effects on immunopathological inflammation following viral infection. Immunology. 152:328-343. https://doi.org/10.1111/imm.0.12771.
Braciale, T. J., and Y. S. Hahn. 2013. Immunity to viruses. Immunol. Rev. 255: 5-12. https://doi.org/10.1111/imr.12109.
Burns, C., A. Cheung, Z. Stark, S. Choo, L. Downie, S. White, R. Conyers, and T. Cole. 2016. A novel presentation of homozygous loss-of-function STAT-1 mutation in an infant with hyperinflammation-A case report and review of the literature. J. Allergy Clin. Immunol. Pract. 4:777-779. https://doi.org/10.1016/j.jaip.2016.02.015.
Casey, K. A., X. Guo, M. A. Smith, S. Wang, D. Sinibaldi, M. A. Sanjuan, L. Wang, G. G. Illei, and W. I. White. 2018. Type I interferon receptor blockade with anifrolumab corrects innate and adaptive immune perturbations of SLE. Lupus Sci. Med. 5. e000286.
Cheng, L., H. Yu, G. Li, F. Li, J. Ma, J. Li, L. Chi, L. Zhang, and L. Su. 2017. Type I interferons suppress viral replication but contribute to T cell depletion and dysfunction during chronic HIV-1 infection. JCI Insight. 2. e94366. https://doi.org/10.1172/jci.insight.94366.
Ciancanelli, M. J., S. X. Huang, P. Luthra, H. Garner, Y. Itan, S. Volpi, F. G. Lafaille, C. Trouillet, M. Schmolke, R. A. Albrecht, et al. 2015. Infectious disease. Life-threatening influenza and impaired interferon amplification in human IRF7 deficiency. Science. 348:448-453. https://doi.org/10.1126/science.aaa1578.
Cotter, C. A., P. L. Earl, L. S. Wyatt, and B. Moss. 2017. Preparation of Cell Cultures and Vaccinia Virus Stocks. Curr. Protoc. Mol. Biol. 117: 16.16.1-16.16.18. https://doi.org/10.1002/cpmb.33.
Crouse, J., G. Bedenikovic, M. Wiesel, M. Ibberson, I. Xenarios, D. Von Laer, U. Kalinke, E. Vivier, S. Jonjic, and A. Oxenius. 2014. Type I interferons protect T cells against NK cell attack mediated by the activating receptor NCR1. Immunity. 40:961-973. https://doi.org/10.1016/j.immuni.2014.05.003.
Dangi, T., Y. R. Chung, N. Palacio, and P. Penaloza-MacMaster. 2020. Interrogating Adaptive Immunity Using LCMV. Curr. Protoc. Immunol. 130. https://doi.org/10.1002/cpim.99
Duncan, C. J., S. M. Mohamad, D. F. Young, A. J. Skelton, T. R. Leahy, D. C. Munday, K. M. Butler, S. Morfopoulou, J. R. Brown, M. Hubank, et al. 2015. Human IFNAR2 deficiency: Lessons for antiviral immunity. Sci. Transl. Med. 7. 307ra154. https://doi.org/10.1126/scitranslmed.aac4227.
Dupuis, S., E. Jouanguy, S. Al-Hajjar, C. Fieschi, I. Z. Al-Mohsen, S. Al-Jumaah, K. Yang, A. Chapgier, C. Eidenschenk, P. Eid, et al. 2003. Impaired response to interferon-alpha/beta and lethal viral disease in human STAT1 deficiency. Nat. Genet. 33:388-391. https://doi.org/10.1038/ng1097
Erickson, A. K., and J. K. Pfeiffer. 2013. Dynamic viral dissemination in mice infected with yellow fever virus strain 17D. J. Virol. 87:12392-12397. https://doi.org/10.1128/JVI.02149-13.
Farrell, P. J., G. C. Sen, M. F. Dubois, L. Ratner, E. Slattery, and P. Lengyel. 1978. Interferon action: two distinct pathways for inhibition of protein synthesis by double-stranded RNA. Proc. Natl. Acad. Sci. USA. 75:5893-5897. https://doi.org/10.1073/pnas.75.12.5893.
Felten, R., F. Scher, F. Sagez, F. Chasset, and L. Arnaud. 2019. Spotlight on anifrolumab and its potential for the treatment of moderate-to-severe systemic lupus erythematosus: evidence to date. Drug Des. Devel. Ther. 13:1535-1543. https://doi.org/10.2147/DDDT.S170969.
Gallimore, A., A. Glithero, A. Godkin, A. C. Tissot, A. Plückthun, T. Elliott, H. Hengartner, and R. Zinkernagel. 1998. Induction and exhaustion of lymphocytic choriomeningitis virus-specific cytotoxic T lymphocytes visualized using soluble tetrameric major histocompatibility complex class I-peptide complexes. J. Exp. Med. 187:1383-1393. https://doi.org/10.1084/jem.187.9.1383.
Gaucher, D., R. Therrien, N. Kettaf, B. R. Angermann, G. Boucher, A. Filali-Mouhim, J. M. Moser, R. S. Mehta, D. R. Drake, III, E. Castro, et al. 2008. Yellow fever vaccine induces integrated multilineage and polyfunctional immune responses. J. Exp. Med. 205:3119-3131. https://doi.org/10.1084/jem.20082292.
Hambleton, S., S. Goodbourn, D. F. Young, P. Dickinson, S. M. Mohamad, M. Valappil, N. McGovern, A. J. Cant, S. J. Hackett, P. Ghazal, et al. 2013. STAT2 deficiency and susceptibility to viral illness in humans. Proc. Natl. Acad. Sci. USA. 110:3053-3058. https://doi.org/10.1073/pnas.1220098110.
Havenar-Daughton, C., G. A. Kolumam, and K. Murali-Krishna. 2006. Cutting Edge: The direct action of type I IFN on CD4 T cells is critical for sustaining clonal expansion in response to a viral but not a bacterial infection. J. Immunol. 176:3315-3319. https://doi.org/10.4049/jimmunol.176.6.3315.
Hernandez, N., I. Melki, H. Jing, T. Habib, S. S. Y. Huang, J. Danielson, T. Kula, S. Drutman, S. Belkaya, V. Rattina, et al. 2018. Life-threatening influenza pneumonitis in a child with inherited IRF9 deficiency. J. Exp. Med. 215:2567-2585. https://doi.org/10.1084/jem.20180628.
Hernandez, N., G. Bucciol, L. Moens, J. Le Pen, M. Shahrooei, E. Goudouris, A. Shirkani, M. Changi-Ashtiani, H. Rokni-Zadeh, E. H. Sayar, et al. 2019. Inherited IFNAR1 deficiency in otherwise healthy patients with adverse reaction to measles and yellow fever live vaccines. J. Exp. Med. 216: 2057-2070. https://doi.org/10.1084/jem.20182295.
Honke, N., N. Shaabani, G. Cadeddu, U. R. Sorg, D. E. Zhang, M. Trilling, K. Klingel, M. Sauter, R. Kandolf, N. Gailus, et al. 2012. Enforced viral replication activates adaptive immunity and is essential for the control of a cytopathic virus. Nat. Immunol. 13:51-57. https://doi.org/10.1038/ni.2169.
Hoyos-Bachiloglu, R., J. Chou, C. N. Sodroski, A. Beano, W. Bainter, M. Angelova, E. Al Idrissi, M. K. Habazi, H. A. Alghamdi, F. Almanjomi, et al. 2017. A digenic human immunodeficiency characterized by IFNAR1 and IFNGR2 mutations. J. Clin. Invest. 127:4415-4420. https://doi.org/10.1172/JCI93486
Kadowaki, N., S. Antonenko, J. Y. Lau, and Y. J. Liu. 2000. Natural interferon alpha/beta-producing cells link innate and adaptive immunity. J. Exp. Med. 192:219-226. https://doi.org/10.1084/jem.192.2.219.
Kallert, S. M., S. Darbre, W. V. Bonilla, M. Kreutzfeldt, N. Page, P. Müller, M. Kreuzaler, M. Lu, S. Favre, F. Kreppel, et al. 2017. Replicating viral vector platform exploits alarmin signals for potent CD8+ T cell mediated tumour immunotherapy. Nat. Commun. 8:15327. https://doi.org/10.1038/ncomms15327.
Kolumam, G. A., S. Thomas, L. J. Thompson, J. Sprent, and K. Murali-Krishna. 2005. Type I interferons act directly on CD8 T cells to allow clonal expansion and memory formation in response to viral infection. J. Exp. Med. 202:637-650. https://doi.org/10.1084/jem.20050821.
Kreins, A. Y., M. J. Ciancanelli, S. Okada, X. F. Kong, N. Ram'1rez-Alejo, S. S. Kilic, J. El Baghdadi, S. Nonoyama, S. A. Mandaviani, F. Ailal, et al. 2015. Human TYK2 deficiency: Mycobacterial and viral infections without hyper-IgE syndrome. J. Exp. Med. 212:1641-1662. https://doi.org/10.1084/jem.20140280.
Le Bon, A., N. Etchart, C. Rossmann, M. Ashton, S. Hou, D. Gewert, P. Borrow, and D. F. Tough. 2003. Cross-priming of CD8+ T cells stimulated by virus-induced type I interferon. Nat. Immunol. 4:1009-1015. https://doi.org/10.1038/ni978.
Li, S., N. L. Sullivan, N. Rouphael, T. Yu, S. Banton, M. S. Maddur, M. McCausland, C. Chiu, J. Canniff, S. Dubey, et al. 2017. Metabolic Phenotypes of Response to Vaccination in Humans. Cell. 169:862-877.e17. https://doi.org/10.1016/j.cell.2017.04.026.
Lin, W. W., E. Moran, R. J. Adams, R. E. Sievers, D. Hauer, S. Godin, and D. E. Griffin. 2020. A durable protective immune response to wild-type measles virus infection of macaques is due to viral replication and spread in lymphoid tissues. Sci. Transl. Med. 12: eaax7799. https://doi.org/10.1126/scitranslmed.aax7799.
Masopust, D., V. Vezys, A. L. Marzo, and L. Lefrancois. 2001. Preferential localization of effector memory cells in nonlymphoid tissue. Science. 291:2413-2417. https://doi.org/10.1126/science.1058867.
McMichael, T. M., Y. Zhang, A. D. Kenney, L. Zhang, A. Zani, M. Lu, M. Chemudupati, J. Li, and J. S. Yount. 2018. IFITM3 Restricts Human Metapneumovirus Infection. J. Infect. Dis. 218:1582-1591. Medzhitov, R., and C. A. Janeway, Jr. 1998. Innate immune recognition and control of adaptive immune responses. Semin. Immunol. 10:351-353. https://doi.org/10.1006/smim.1998.0136.
Minegishi, Y., M. Saito, T. Morio, K. Watanabe, K. Agematsu, S. Tsuchiya, H. Takada, T. Hara, N. Kawamura, T. Ariga, et al. 2006. Human tyrosine kinase 2 deficiency reveals its requisite roles in multiple cytokine signals involved in innate and acquired immunity. Immunity. 25:745-755. https://doi.org/10.1016/j.immuni.2006.09.009.
Moens, L., L. Van Eyck, D. Jochmans, T. Mitera, G. Frans, X. Bossuyt, P. Matthys, J. Neyts, M. Ciancanelli, S. Y. Zhang, et al. 2017. A novel kindred with inherited STAT2 deficiency and severe viral illness. J. Allergy Clin. Immunol. 139:1995-1997.e9. https://doi.org/10.1016/j.jaci.2016.10.033.
Mueller, S. N., and R. Ahmed. 2009. High antigen levels are the cause of T cell exhaustion during chronic viral infection. Proc. Natl. Acad. Sci. USA. 106: 8623-8628. https://doi.org/10.1073/pnas.0809818106.
Müller, U., U. Steinhoff, L. F. Reis, S. Hemmi, J. Pavlovic, R. M. Zinkernagel, and M. Aguet. 1994. Functional role of type I and type II interferons in antiviral defense. Science. 264:1918-1921. https://doi.org/10.1126/science.8009221.
Norris, B. A., L. S. Uebelhoer, H. I. Nakaya, A. A. Price, A. Grakoui, and B. Pulendran. 2013. Chronic but not acute virus infection induces sustained expansion of myeloid suppressor cell numbers that inhibit viral specific T cell immunity. Immunity. 38:309-321. https://doi.org/10.1016/j.immuni.2012.10.022.
Penaloza-MacMaster, P. 2017. CD8 T-cell regulation by T regulatory cells and the programmed cell death protein 1 pathway. Immunology. 151(2): 146-153. https://doi.org/10.1111/imm.12739.
Penaloza-MacMaster, P., A. O. Kamphorst, A. Wieland, K. Araki, S. S. Iyer, E. E. West, L. O'Mara, S. Yang, B. T. Konieczny, A. H. Sharpe, et al. 2014. Interplay between regulatory T cells and PD-1 in modulating T cell exhaustion and viral control during chronic LCMV infection. J. Exp. Med.211(9):1905-1918. https://doi.org/10.1084/jem.20132577.
Penaloza-MacMaster, P., D. L. Barber, E. J. Wherry, N. M. Provine, J. E. Teigler, L. Parenteau, S. Blackmore, E. N. Borducchi, R. A. Larocca, K. B. Yates, et al. 2015. Vaccine-elicited CD4 T cells induce immunopathology after chronic LCMV infection. Science. 347:278-282. https://doi.org/10.1126/science.aaa2148.
Penaloza MacMaster, P., J. L. Shields, Q. A. Alayo, C. Cabral, J. Jimenez, J. Mondesir, A. Chandrashekar, J. M. Cabral, M. Lim, M. J. Iampietro, et al. 2017. Development of novel replication-defective lymphocytic choriomeningitis virus vectors expressing SIV antigens. Vaccine. 35:1-9. https://doi.org/10.1016/j.vaccine.2016.11.063.
Pulendran, B., and R. Ahmed. 2011. Immunological mechanisms of vaccination. Nat. Immunol. 12:509-517. https://doi.org/10.1038/ni.2039.
Pulendran, B., S. Li, and H. I. Nakaya. 2010. Systems vaccinology. Immunity. 33:516-529. https://doi.org/10.1016/j.immuni.2010.10.006
Pulendran, B., J. Z. Oh, H. I. Nakaya, R. Ravindran, and D. A. Kazmin. 2013. Immunity to viruses: learning from successful human vaccines. Immunol. Rev. 255:243-255. https://doi.org/10.1111/imr.12099.
Querec, T. D., R. S. Akondy, E. K. Lee, W. Cao, H. I. Nakaya, D. Teuwen, A. Pirani, K. Gernert, J. Deng, B. Marzolf, et al. 2009. Systems biology approach predicts immunogenicity of the yellow fever vaccine in humans. Nat. Immunol. 10:116-125. https://doi.org/10.1038/ni.1688.
Quigley, M., F. Pereyra, B. Nilsson, F. Porichis, C. Fonseca, Q. Eichbaum, B. Julg, J. L. Jesneck, K. Brosnahan, S. Imam, et al. 2010. Transcriptional analysis of HIV-specific CD8+ T cells shows that PD-1 inhibits T cell function by upregulating BATF. Nat. Med. 16:1147-1151. https://doi.org/10.1038/nm.2232.
Reizis, B., A. Bunin, H. S. Ghosh, K. L. Lewis, and V. Sisirak. 2011. Plasmacytoid dendritic cells: recent progress and open questions. Annu. Rev. Immunol. 29:163-183. https://doi.org/10.1146/annurev-immuno1-031210-101345.
Sandler, N. G., S. E. Bosinger, J. D. Estes, R. T. Zhu, G. K. Tharp, E. Boritz, D. Levin, S. Wijeyesinghe, K. N. Makamdop, G. Q. del Prete, et al. 2014. Type I interferon responses in rhesus macaques prevent SIV infection and slow disease progression. Nature. 511:601-605. https://doi.org/10.1038/nature13554.
Schoggins, J. W. 2019. Interferon-Stimulated Genes: What Do They All Do? Annu. Rev. Virol. 6:567-584. https://doi.org/10.1146/annurev-virology-092818-015756.
Shahni, R., C. M. Cale, G. Anderson, L. D. Osellame, S. Hambleton, T. S. Jacques, Y. Wedatilake, J. W. Taanman, E. Chan, W. Qasim, et al. 2015. Signal transducer and activator of transcription 2 deficiency is a novel disorder of mitochondrial fission. Brain. 138:2834-2846. https://doi.org/10.1093/brain/awv182.
Stark, G. R., I. M. Kerr, B. R. Williams, R. H. Silverman, and R. D. Schreiber. 1998. How cells respond to interferons. Annu. Rev. Biochem. 67:227-264. https://doi.org/10.1146/annurev.biochem.67.1.227.
Tanaka, Y., T. Takeuchi, M. Okada, T. Ishii, H. Nakajima, S. Kawai, T. Nagashima, N. Hayashi, L. Wang, and R. Tummala. 2020. Safety and tolerability of anifrolumab, a monoclonal antibody targeting type I interferon receptor, in Japanese patients with systemic lupus erythematosus:
A multicenter, phase 2, open-label study. Mod. Rheumatol. 30: 101-108. https://doi.org/10.1080/14397595.2019.1583833.
Teijaro, J. R., C. Ng, A. M. Lee, B. M. Sullivan, K. C. Sheehan, M. Welch, R. D. Schreiber, J. C. de la Torre, and M. B. Oldstone. 2013. Persistent LCMV infection is controlled by blockade of type I interferon signaling. Science. 340:207-211. https://doi.org/10.1126/science.1235214.
Torriani, F. J., M. Rodriguez-Torres, J. K. Rockstroh, E. Lissen, J. Gonzalez-Garc'1a, A. Lazzarin, G. Carosi, J. Sasadeusz, C. Katlama, J. Montaner, et al; APRICOT Study Group. 2004. Peginterferon Alfa-2a plus ribavirin for chronic hepatitis C virus infection in HIV-infected patients. N. Engl. J. Med. 351:438-450. https://doi.org/10.1056/NEJMoa040842.
Wang, Y., Y. R. Chung, S. Eitzinger, N. Palacio, S. Gregory, M. Bhattacharyya, and P. Penaloza-MacMaster. 2019. TLR4 signaling improves PD-1 blockade therapy during chronic viral infection. PLoS Pathog. 15. e1007583. https://doi.org/10.1371/journal.ppat. 1007583.
Welsh, R. M., K. Bahl, H. D. Marshall, and S. L. Urban. 2012. Type 1 interferons and antiviral CD8 T-cell responses. PLoS Pathog. 8. e1002352. https://doi.org/10.1371/journal.ppat.1002352.
Wherry, E. J., V. Teichgr\ddot{ }aber, T. C. Becker, D. Masopust, S. M. Kaech, R. Antia,
U. H. von Andrian, and R. Ahmed. 2003. Lineage relationship and protective immunity of memory CD8 T cell subsets. Nat. Immunol. 4: 225-234. https://doi.org/10.1038/ni889.
Wilson, E. B., D. H. Yamada, H. Elsaesser, J. Herskovitz, J. Deng, G. Cheng, B. J. Aronow, C. L. Karp, and D. G. Brooks. 2013. Blockade of chronic type I interferon signaling to control persistent LCMV infection. Science. 340: 202-207. https://doi.org/10.1126/science.1235208.
Xu, H. C., M. Grusdat, A. A. Pandyra, R. Polz, J. Huang, P. Sharma, R. Deenen, K. K\ddot{ }ohrer, R. Rahbar, A. Diefenbach, et al. 2014. Type I interferon protects antiviral CD8+ T cells from NK cell cytotoxicity. Immunity. 40: 949-960. https://doi.org/10.1016/j.immuni.2014.05.004.
Zuniga, E. I., L. Y. Liou, L. Mack, M. Mendoza, and M. B. Oldstone. 2008. Persistent virus infection inhibits type I interferon production by plasmacytoid dendritic cells to facilitate opportunistic infections. Cell Host Microbe. 4:374-386. https://doi.org/10.1016/j.chom.2008.08.016.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations that is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for enhancing an immune response in a subject in need thereof, the method comprising administering a pharmaceutical composition comprising an effective amount of a therapeutic agent that blocks the activity of interferon and a pharmaceutical composition comprising an effective amount of an antigen.

2. The method of claim 1, wherein the method vaccinates the subject against an infectious agent (e.g., a virus or a bacteria) or disease (e.g., cancer) that comprises the antigen.

3. The method of claim 1, wherein the pharmaceutical composition comprising the effective amount of the antigen is a viral vaccine.

4. The method of claim 3, wherein the viral vaccine is selected from the group consisting of an influenza vaccine, a hepatitis A vaccine, a hepatitis B vaccine, a human papilloma virus vaccine, a zoster vaccine, a smallpox vaccine, a measles vaccine, a rabies vaccine, a poliovirus vaccine, a Japanese encephalitis vaccine, a rubella vaccine, a rotavirus vaccine, a yellow fever vaccine, a varicella virus vaccine, an HIV vaccine, and any combination thereof.

5. The method of claim 1, wherein the pharmaceutical composition comprising the effective amount of the antigen is an anti-cancer vaccine.

6. The method of claim 1, wherein the therapeutic agent that blocks the activity of IFN-I comprises an antibody.

7. The method of claim 1, wherein the therapeutic agent that blocks the activity of IFN-I comprises an IFN-I receptor binding antibody.

8. The method of claim 1, wherein the therapeutic agent that blocks the activity of IFN-I comprises αIFNAR1 clone MAR1-5A3.

9. The method of claim 1, wherein the therapeutic agent that blocks the activity of IFN-I comprises anifrolumab.

10. The method of claim 1, wherein the pharmaceutical composition comprising the effective amount of the therapeutic agent that blocks the activity of the IFN-I pathway is admixed with the pharmaceutical composition comprising the effective amount of the antigen prior to administering to the subject.

11. The method of claim 1, wherein the pharmaceutical composition comprising the effective amount of the therapeutic agent that blocks the activity of IFN-I is administered separately from the pharmaceutical composition comprising the effective amount of the antigen.

12. The method of claim 1, wherein the pharmaceutical composition comprising the effective amount of the therapeutic agent that blocks the activity of IFN-I is administered within 24 hours, within 48 hours, or within 72 hours of administering the pharmaceutical composition comprising the effective amount of the antigen.

13. The method of claim 1, wherein the improved immune response comprises one or more of the following, optionally after the subject further subsequently is administered the antigen and/or the subject is later challenged with an infectious agent (e.g. a virus or a bacteria) or disease (e.g., cancer) comprising the antigen: improved vaccine immunogenicity and/or enhanced immune protection; an increase in antigen-specific CD8 T-cells; an increase in antigen-specific CD4 T-cells; an increase in antigen-specific B cells or antibodies; an increase in heterologous antibodies; a lack of, or reduction in, disease symptoms; and/or a lack of, or reduction in the antigen.

14. An immunogenic composition comprising: an effective amount of an antigen; and a therapeutic agent that blocks the activity of IFN-1.

15. The immunogenic composition of claim 14, wherein the immunogenic composition comprises a vaccine against an infectious agent that comprises the antigen.

16. The composition of claim 14, wherein the composition comprises a vaccine selected from the group consisting of: an influenza vaccine, a hepatitis A vaccine, a hepatitis B vaccine, a human papilloma virus vaccine, a zoster vaccine, a smallpox vaccine, a measles vaccine, a rabies vaccine, a poliovirus vaccine, a Japanese encephalitis vaccine, a rubella vaccine, a rotavirus vaccine, a yellow fever vaccine, a varicella virus vaccine, an HIV vaccine, and other replicating viral vaccines, and combinations thereof.

17. The composition of claim 14, wherein the composition comprises an anti-cancer vaccine.

18. The composition of claim 14, wherein the therapeutic agent that blocks the activity of IFN-I comprises an antibody.

19. The composition of claim 14, wherein the therapeutic agent that blocks the activity of IFN-I comprises an IFN-I receptor binding antibody.

20. The composition of claim 14, wherein the therapeutic agent that blocks the activity of IFN-I comprises αIFNAR1 clone MAR1-5A3.

21. The composition of claim 14, wherein the therapeutic agent that blocks the activity of IFN-I comprises anifrolumab.

22. A kit comprising: (a) an antigen, optionally wherein the kit comprises a vaccine; and (b) an IFN-I blocking agent.

23. The kit of claim 22, wherein the kit comprises a viral vaccine, and wherein the IFN-I blocking agent comprises an IFN-I receptor binding antibody.

24. The kit of claim 22, wherein the kit comprises an anti-cancer vaccine, and wherein the IFN-I blocking agent comprises an IFN-I receptor binding antibody.
